(19) United States
(12) Patent Application Publication
(10) Pub. No.: US 2014/0342931 A1
(43) Pub. Date: Nov. 20, 2014

(54) METHOD FOR PREDICTING RESPONSE TO ENDOCRINE THERAPY

(71) Applicants: Rene Houtman, Culemborg (NL); Rob Ruijtenbeek, Utrecht (NL); Rob Michalides, Amsterdam (NL)

(72) Inventors: Rene Houtman, Culemborg (NL); Rob Ruijtenbeek, Utrecht (NL); Rob Michalides, Amsterdam (NL)

(73) Assignees: PAMGENE B.V., 's-Hertogenbosch (NL); STICHTING HET NEDERLANDS KANKER INSTITUUT, Amsterdam (NL)

(21) Appl. No.: 14/344,676

(22) PCT Filed: Sep. 14, 2012
(86) PCT No.: PCT/EP2012/068153

§ 371 Date: Mar. 13, 2014

(30) Foreign Application Priority Data
Sep. 16, 2011 (EP) 11181717.7

(51) Int. Cl. G01N 33/574

(52) U.S. Cl. CPC G01N 33/57415; G01N 2500/10; G01N 2333/723
USPC 506/9

(57) ABSTRACT

The present invention relates to a method for predicting the response of a patient diagnosed with breast cancer to treatment with an endocrine therapy drug, comprising the steps of: (a) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of one or more added phosphatases, (b) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of added estradiol, and, (c) predicting from said binding profiles the response of said patient to endocrine drug therapy. The present invention also relates to variant methods hereof and methods for predicting the response of a patient diagnosed with breast cancer to drug treatment and methods for individualized endocrine therapy of a patient diagnosed with an endocrine related disease. The invention also relates to arrays and kits for carrying out these methods.

### FIELD OF THE INVENTION

The present invention relates to methods and kits for predicting the response of patients to endocrine therapy, more particularly of predicting the response of patients diagnosed with breast cancer.

### BACKGROUND

Breast cancer is a cancer that starts in the cells of the breast in women and men. Worldwide, breast cancer is the second most common type of cancer after lung cancer (about 10% of all cancer incidences) and the fifth most common cause of cancer death.

Due to the high impact of breast cancer an early diagnosis of breast cancer is essential, especially since this improves the survival rate of breast cancer patients. Therefore in breast cancer, regular mammography and early diagnosis is of high importance. This increases the chances that the lymph nodes are not infiltrated, that the tumor can be surgically removed and local or regional therapy (radiation therapy) is sufficient.

In many cases of early and advanced breast cancer local or regional treatment is insufficient. In those cases, establishing the second line therapy most suited for each breast cancer patient is essential. After removal of (part) of the breast, systemic treatment like chemotherapy or targeted therapy is used. With new drugs, especially those targeting kinases, selection of patients using molecular diagnostics appears to be critical for success. Biomarkers like the estrogen receptor (ER), the progesterone receptor (PR) or the human epidermal growth factor receptor 2 (HER2) play an important role in deciding whether hormone therapy, Herceptin or another drug is included in the treatment of choice.

Determining the type of breast cancer is therefore important for providing the most suited treatment of the patient. It is known that for early and advanced breast cancer both in pre- and postmenopausal women, Tamoxifen or another anti-estrogen like raloxifene, lasofoxifene or bazedoxifene, is a suited treatment for an estrogen receptor positive (ER+) and/or an progesterone receptor positive (PR+) breast tumor. Tamoxifen is an anti-estrogen from the group of SERMs (Selective Estrogen Receptor Modulator).

Recently, aromatase inhibitors have become the drugs of choice for treatment of breast cancer in post-menopausal ER+ or PR+ women. Aromatase inhibitors prevent the formation of estrogens by inhibition of enzymes that catalyze the conversion of androsterons to estrogen. By blocking the action of the enzyme aromatase, no more estrogens are produced in the body.

Human epidermal growth factor receptor 2 positive (HER2+) breast cancer is currently treated with Herceptin. For breast tumors that are estrogen receptor negative, progesterone receptor negative and HER2 negative, no targeted therapy is available and in general prognosis is poor.

For determining whether a breast tumor is either ER positive or negative, HER2 positive or negative and/or PR positive or negative usually immunohistochemical, PCR or FISH methods are used. These methods localize the estrogen, human epidermal growth factor or progesterone receptors in the tumor cells using antibodies binding specifically to the estrogen, human epidermal growth factor or progesterone receptors. However, these immunohistochemical measurements are not well standardized yet and their reliability to predict hormone therapy responses is limited.

The presence of estrogen receptors is the best indicator of response to anti-estrogen agents such as tamoxifen. However, 30% to 40% of women with estrogen receptor positive breast cancer will develop distant metastases and die despite tamoxifen treatment, which percentage is even higher for ER+ PR− (60%).

Consequently, there remains need for methods that provide a fast and accurate measurement of the estrogen, human epidermal growth factor or progesterone receptor status in breast tumors. These methods would enable the identification of the type of breast cancer at an early stage, and more specifically provide an early determination of the most suited treatment of the breast cancer patient.

Nuclear receptors (NRs) regulate gene expression levels by transactivation, and thereby perform two functions, i.e. gene promotor binding and recruitment of coregulators. Modulation of NR activity is usually quantitatively analyzed by measurement of target gene transcription or downstream events. These parameters are however the net result of the NR interactions with individual coregulators, and lack the resolution to explain different outcomes that are the result of subtle alterations in these interactions. Thus far, studying nuclear receptor interactions with coregulators has been a challenge.

Conventional methods providing NR-coregulator interaction data are intermolecular FRET, Y2H, phage display and colocalization studies in fluorescence microscopy, each with their own limitations. The present invention aims at developing useful methods and arrays that can assess full length estrogen receptor function, i.e. coregulator interaction, in a high throughput manner.

The family of coregulator proteins consists of coactivators and corepressors. They can form a physical and functional bridge between the nuclear receptor and the gene transcription machinery. Coactivators interact with nuclear receptors via small hydrophobic and amphipathic α helical peptide sequences with the common signature motif, LxxLL (L=leucine, x=any amino acid). Crystallography studies have shown that this motif, also called nuclear receptor box (NR-box), binds to a hydrophobic cleft on the NR Ligand Binding Domain (LBD) surface, under control of the ligand. Coactivators can contain one or multiple NR-boxes within their central nuclear receptor interaction domain. Although the three leucines in the LxxLL-motif are conserved, the amino acid residues flanking this sequence vary. These residues determine NR box recognition for a particular nuclear receptor. NR-coregulator interaction is mainly dictated by ligand, which induces a conformational change in the LBD. Apart from the ligand, post-translational modifications (PTM) also play a large role in NR transactivation and can cause differential response to ligands. It is therefore reasonable to assume that PTMs can also play a role in coregulator recruitment.

The most widely studied group of estrogen receptor alpha (ERα) coregulators includes the p160 protein family, consisting of three members: NCOA1 (SRC-1), NCOA2 (SRC-2) and NCOA3 (SRC-3) (21-23). These coactivators are recruited by ERα upon binding of the natural ligand estradiol. Knockout studies in mice and rats have shown that these coactivators have important endocrine functions in processes, such as development of the brain and the reproductive system. And although they can partially compensate for loss of family members, the mouse phenotypes demonstrate that they have specificity. Moreover, AlB1 gene amplification and elevated expression was discovered in a subset of ERα-positive breast cancer (24;25). Endocrine therapy, which aims for inactivation of ERα, uses competitive estrogen antagonists (e.g. tamoxifen) or aromatase inhibitors that block estrogen synthesis. This prevents the formation of the coactivator binding surface on ERα. A group of patients does not respond to endocrine therapy, because ERα remains transcriptionally active. This indicates that ERα activity is controlled by additional factors which are largely unknown. One factor that is associated with resistance to tamoxifen is phosphorylation of ERα Serine 305 by protein kinase A. This post-translational modification affects receptor function by a conformational change that alters binding to SRC-1. Since ERα transcriptional activity is defined by interaction of the receptor with a multitude of different coregulators, we decided to functionally analyze the effect of Ser305-P, i.e. interaction with a broader panel of coregulators.

The present invention aims at developing methods, arrays, kits and uses for predicting the response of patients diagnosed with breast cancer to treatment with endocrine therapy. Also, the present invention aims at developing methods, arrays, kits and uses for predicting the response of such patients to drug treatment. Further the present invention aims at providing methods, kits, arrays and uses for individualized endocrine therapy of a patient diagnosed with an endocrine related disease.

### SUMMARY OF THE INVENTION

The present invention relates to a method for predicting the response of a patient diagnosed with breast cancer to treatment with an endocrine therapy drug, comprising the steps of:

(a) measuring on the basis of a sample, obtained from said patient, the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of one or more added phosphatases,

(b) measuring on the basis of a sample, obtained from said patient, the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of added estradiol, and,

(c) predicting from said binding profiles the response of said patient to endocrine drug therapy.

More particularly, the method according to the present invention further comprises a step (c) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol and one or more added phosphatases, followed by a step (d) of predicting from the binding profiles the response of said patient to endocrine drug therapy.

More particularly, said endocrine therapy and endocrine therapy drug is an estrogen receptor therapy and estrogen receptor therapy drug. More particularly, said one or more co-regulator proteins or functional parts thereof are immobilized onto a solid support. The present invention also relates to a method for predicting the response of a patient diagnosed with breast cancer to drug treatment, comprising the steps of:

(a) measuring the estrogen receptor binding activity of a sample, obtained from the breast cancer tumor from said patient, thereby providing the estrogen receptor binding profiles in the presence and absence of one or more added phosphatases, estradiol and optionally a drug using the markers as listed in Table 1 selected from the group comprising SEQ ID NO 1 to 154; and,

(b) predicting from said estrogen receptor binding profiles the response of said patient to drug therapy.

Further the present invention relates to a method for individualized estrogen receptor therapy of a patient diagnosed with an estrogen receptor related disease comprising the steps of:

(a) measuring the activity of the estrogen receptor, obtained from a sample from said patient, generating an activity profile, said activity profile comprising the activity of the estrogen receptor from said patient on one or more co-regulator proteins or functional parts thereof in the presence and absence of one or more added phosphatases,

(b) measuring the activity of the estrogen receptor, obtained from a sample from said patient, generating an activity profile, said activity profile comprising the activity of the estrogen receptor from said patient on one or more co-regulator proteins or functional parts thereof in the presence and absence of added estradiol, and,

(c) predicting from said activity profiles the response and optimum dose of said estrogen receptor therapy to said patient.

More particularly, the method for individualized estrogen receptor therapy according to the present invention further comprises a step (c) measuring the activity of the estrogen receptor, obtained from a sample from said patient, generating an activity profile, said activity profile comprising the activity of the estrogen receptor from said patient on one or more co-regulator proteins or functional parts thereof in the presence and absence of added estradiol and one or more added phosphatases, followed by a step (d) of predicting from said activity profiles the response and optimum dose of said estrogen receptor therapy to said patient.

More particularly, said one or more co-regulator proteins or functional parts thereof are immobilized onto a solid support.

Still further, the present invention relates to a method for individualized endocrine therapy of a patient diagnosed with an endocrine related disorder comprising the steps of:

(a) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of one or more added phosphatases,

(b) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of added ligand of said nuclear receptor, and,

(c) predicting from said activity profiles the response and optimum dose of said endocrine therapy to said patient.

More particularly, the method for individualized endocrine therapy according to the present invention further comprises a step (c) measuring the binding of the nuclear receptor, obtained from a sample from said endocrine disease from said patient, generating a binding activity profile, said binding activity profile comprising the binding of the nuclear receptor from said patient on one or more immobilized co-regulator proteins or functional parts thereof in the presence and absence of added estradiol and one or more added phosphatases, followed by a step (d) of predicting from the binding profiles the response of said patient to endocrine drug therapy.

More particularly, said endocrine therapy and endocrine therapy drug is an estrogen receptor therapy and estrogen receptor therapy drug. More particularly, said one or more co-regulator proteins or functional parts thereof are immobilized onto a solid support.

Further the present invention relates to an array for carrying out the method of the invention, said array comprising immobilized proteins, peptides or peptide mimetics comprising estrogen receptor binding sites present in at least two peptide markers as listed in Table 1 selected from the group comprising SEQ ID NO 1 to 154.

The present invention also relates to a kit for predicting the response of a patient diagnosed with breast cancer to drug treatment, comprising at least one array of the invention.

### DESCRIPTION OF THE FIGURES

All sequence ID numbers referred to in the following figure descriptions and examples are listed in Tables 1 and 2.

FIG. 1 shows the effect of serine 305 phosphorylation on ERα-coregulator binding. U2OS cells were transfected with wildtype (WT), single (305A) or double (236A-305A) serine mutant full-length ERα tagged with YFP/CFP by Western blot. Cells were stimulated with (+) or without cAMP to induce PKA-mediated receptor phosphorylation.

FIG. 2 shows the dose dependent 17-β-Estradiol (E2)—modulated binding of wildtype or ERαY/C 305A from control or cAMP-stimulated cells with NCOA1_{—}677_{—}700 (IDNR13) coregulator peptide as outlined in the method according to WO 2008/028978

FIG. 3 shows the tamoxifen-induced modulation of ERα-coregulator binding in the method according to WO 2008/028978. U2OS cells were transfected with ERαY/C wildtype (WT) or ERαSer305A-Y/C (305A). Cells were stimulated with (+) or without cAMP and dose dependent 4-hydroxy-Tamoxifen (4-OHT)—modulated binding to NCOA1_{—}677_{—}700 (IDNR13) by wildtype or ERα305A-Y/C in vehicle or cAMP-stimulated cells.

FIG. 4 shows the dose response curve derived tamoxifen (4-OHT) potency of binding modulation of wildtype ERα from control vs. cAMP-stimulated cells to coregulator peptides the tamoxifen-induced modulation of ERα-coregulator binding in the method according to WO 2008/028978 on 52 coregulator peptides as outlined in table 2.

FIG. 5 shows A. Immunohistochemistry of total ERα and ERαSer305-P status in the sample of a S305P−(tumor A) or+(tumor B) patient.

FIG. 6 shows the binding of the estrogen receptor from breast cancer tumors on coregulator peptides according to WO 2008/028978 of tumor A and B after treating lysates without (vehicle) or with (E2) 17-β-estradiol.

FIG. 7 shows a Western blot analysis of total ERα and ERαSer305-P status in the sample of a S305P−(tumor A) or+(tumor B) patient before (−) and after (+) phosphatase treatment of tumor lysates.

FIG. 8 shows coregulator binding according to the method of WO 2008/028978 in samples from S305P−or+patients, untreated (grey) or after phosphatase treatment (black). In the absence (vehicle) or presence of E2 (saturating concentration) or 4-OHT at the EC50 concentration.

### DETAILED DESCRIPTION OF THE INVENTION

Before the present method and devices used in the invention are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

The present invention relates to a method for predicting the response of a patient diagnosed with breast cancer to treatment with an endocrine therapy drug, more particularly estrogen receptor therapy drug, comprising the steps of:

(a) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of one or more added phosphatases,

(b) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of added estradiol, and,

(c) predicting from said binding profiles the response of said patient to endocrine drug therapy.

More particularly, the method for predicting the response of a patient diagnosed with breast cancer to treatment with an estrogen receptor therapy drug, comprising the steps of:

(a) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of one or more added phosphatases,

(b) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol, and,

(c) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol and one or more added phosphatases, and,

(d) predicting from said binding profiles the response of said patient to estrogen receptor drug therapy.

The present invention also relates to a method for predicting the response of a patient diagnosed with breast cancer to treatment with an endocrine therapy drug, more particularly estrogen receptor therapy drug, comprising the steps of:

(a) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of one or more added phosphatases,

(b) measuring the binding of the estrogen receptor, obtained from a breast cancer tumor from said patient, generating a binding profile, said binding profile comprising the binding of the estrogen receptor from said patient on one or more co-regulator proteins or functional parts thereof in the presence and absence of added estradiol,

(c) measuring the binding of the estrogen receptor, obtained from a breast cancer tumor from said patient, generating a binding profiling, said binding profile comprising the binding of the estrogen receptor from said patient on one or more co-regulator proteins or functional parts thereof in the presence and absence of one or more added drugs, and,

(d) predicting from said binding profiles the response of said patient to endocrine drug therapy.

More particularly, the method for predicting the response of a patient diagnosed with breast cancer to treatment with an estrogen receptor therapy drug, comprising the steps of:

(a) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of one or more added phosphatases,

(b) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol, and,

(c) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol and one or more added phosphatases,

(d) measuring on the basis of a sample the binding of the estrogen receptor, obtained from a breast cancer tumor from said patient, generating a binding profiling, said binding profile comprising the binding of the estrogen receptor from said patient on one or more co-regulator proteins or functional parts thereof in the presence and absence of one or more added drugs, and,

(e) predicting from said binding profiles the response of said patient to estrogen receptor drug therapy.

These drugs are preferably endocrine drugs. Said endocrine therapy drug, more particularly estrogen receptor therapy drug, may for instance consist of or comprises tamoxifen, raloxifene, lasofoxifene or bazedoxifene or aromatase inhibitors.

Said binding profiling may be determined on proteins, peptides or peptide mimetics immobilized on a solid support, and preferably immobilized on a porous solid support as detailed below.

Preferably said peptides are at least two peptides as listed in Table 1 selected from a group consisting of any of the SEQ ID NO 1 to 154.

More preferably said peptides are at least SEQ ID NO 1 to 52 as listed in Table 2.

The endocrine system is a system of glands, each of which secretes a type of hormone directly into the bloodstream to regulate the body. Hormones released from endocrine tissue into the bloodstream travel to target tissue and generate a response. Hormones regulate various human functions, including metabolism, growth and development, tissue function, and mood.

Endocrine therapy refers to a treatment that adds, blocks, or removes hormones. To slow or stop the growth of certain cancers (such as prostate and breast cancer), synthetic hormones or other drugs may be given to block the body's natural hormones. This is also referred to as hormonal therapy, hormone therapy, and hormone treatment. Estrogen is one of the main female hormones regulating reproduction. In younger women, the ovaries produce estrogen. After menopause, they stop, and a woman will no longer have periods. But postmenopausal women still produce a limited amount of the hormone. Another part of the endocrine system, the adrenal glands, makes a hormone called androgen and aromatase, an enzyme that is produced by fat cells, can convert androgen into estrogen. Accordingly, a common endocrine therapy for breast cancer is referred to as estrogen receptor therapy, where the active compounds target the estrogen receptor thereby blocking the body's natural estrogen hormone. By removing or reducing the estrogen available to the tumor, better survival rates for women with estrogen receptor-positive cancer are obtained, whether its confined to the breast tissue or has spread, or metastasized, to other parts of the body. Accordingly, in a particular embodiment said endocrine therapy refers to estrogen receptor therapy.

Phosphatase activity is referred to as the activity of protein phosphatases. A phosphatase is a generic name for all enzymes able to remove a phosphate group from a substrate by hydrolysing phosphoric acid monoesters into a phosphate ion and a molecule with a free hydroxyl group. This action is directly opposite to that of phosphorylases and kinases, which attach phosphate groups to their substrates by using energetic molecules like ATP. Protein phosphatases (PPs) are the primary effectors of dephosphorylation and can be grouped into three main classes based on sequence, structure and catalytic function. The largest class of PPs is the phosphoprotein phosphatase (PPP) family comprising PP1, PP2A, PP2B, PP4, PPS, PP6 and PP7, and the protein phosphatase Mg²⁺- or Mn²⁺-dependent (PPM) family, composed primarily of PP2C. The protein Tyr phosphatase (PTP) super-family forms the second group, and the aspartate-based protein phosphatases the third.

In particular embodiments the methods according to the present invention provide that said one or more added phosphatases are chosen from the protein phosphatase Mg²⁺- or Mn²⁺-dependent (PPM) family or an alkaline phosphatase and more particularly lambda phosphatase

As used in the present invention, the term “sample” refers to a sample obtained from an organism (patient) such as human or from components (e.g. tissue or cells) of such an organism. Said sample is preferably obtained from a patient diagnosed with breast cancer or any other endocrine related disease such as detailed in the methods below and may preferably need to be derived from the tumor tissue of said patient. More preferably said sample is a breast tumor tissue biopsy, fine needle biopsy, fine needle aspiration biopsy, core needle biopsy, vacuum assisted biopsy, open surgical biopsy or material from a resected tumor. Said sample is thereby referred to as a ‘clinical sample’ which is a sample derived from a breast cancer patient.

Said tumor tissue sample is preferably a fresh or a fresh frozen sample.

More preferably, said sample refers to a lysate of a breast tumor tissue obtained through tumor tissue biopsy, fine needle biopsy, fine needle aspiration biopsy, core needle biopsy, open surgical biopsy or material from a resected tumor. Alternatively said sample may be obtained from specific breast tumor cell lines and in particular cell lysates thereof.

Alternatively said sample may be derived from a tumor sample that has been cultured in vitro for a limited period of time.

In a preferred embodiment of the present invention said sample is a sample that has undergone a preparation step prior to the steps according to the method of the present invention. Preferably said preparation step is a step where the protein kinases present in said sample are released from the tissue by lysis. Additionally the kinases in the sample may be stabilized, maintained, enriched or isolated, and the measurement of the kinase activity as performed in step (a) occurs on the enriched or isolated protein kinase sample. By first enriching protein kinases in the sample or isolating protein kinases from the sample the subsequent measurement of the kinase activity will occur in a more efficient and reliable manner. Also the clarity and intensity of the obtained phosphorylation signal will be increased as certain contaminants are being removed during the enriching or isolating step.

In another embodiment according to the present invention, peptide markers as listed in table 1 may be at least 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153 or 154 of the peptide markers listed in Table 1. In another embodiment according to the present invention, peptide markers as listed in table 2 may be at least 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 of the peptide markers listed in Table 2.

The term “peptide markers” in the context of the present invention refers to the fact that the peptides as listed in Table 1 can be preferably used according to the methods of the present invention Therefore the present invention is not limited to the use of peptides identical to any of these peptide markers as listed in Table 1 as such. The skilled person may easily on the basis of the peptide markers listed in Table 1 design variant peptides compared to the specific peptides in said Table and use such variant peptides having nuclear receptor binding sites common to said peptide markers as listed in Table 1. These variant peptides may have one or more (2, 3, 4, 5, 6, 7, etc.) amino acids more or less than the given peptides and may also have amino acid substitutions (preferably conservative amino acid substitutions) as long as these variant peptides retain at least, preferably one or more, of the nuclear receptor binding sites of said original peptides as listed in said table. Further the skilled person may also easily carry out the methods according to the present invention by using proteins (full length or N- or C-terminally truncated) comprising the amino acid regions of the “peptide markers” listed in Table 1 as sources for studying the nuclear receptor binding sites present in the amino acid regions of the peptides listed in Table 1.

A person skilled in the art will appreciate that the nuclear receptor binding sites present in a single peptide marker as listed in Table 1 enable the methods of the invention. However, when the number of peptide markers as listed in Table 1 increases, so will increase the specificity and sensitivity of the method according to the present invention.

As detailed in the Examples section and as illustration of the present invention, the present inventors applied an array on which a set of peptides representing coregulator NR-box sequences are immobilized. This format allows for high throughput, in vitro functional analysis of ERα, i.e. coregulator interaction, and modulation by ligand and receptor phosphorylation. We can detect differences in binding upon one single post-translational modification: phosphorylation of ERα Serine 305. In a series of experiments, the complexity of the sample was increased from recombinant ERα to ERα from crude lysates of transfected cells, from ERα-positive cell line MCF-7 to primary breast tumors.

Alternatively the present invention relates to a method for predicting the response of a patient diagnosed with breast cancer to drug treatment, comprising the steps of:

(a) measuring the estrogen receptor binding activity of a sample, obtained from the breast cancer tumor from said patient, thereby providing the estrogen receptor binding profiles in the presence and absence of one or more added phosphatases, estradiol and optionally drug using the markers as listed in Table 1 selected from the group comprising SEQ ID NO 1 to 154; and,

(b) predicting from said estrogen receptor binding profiles the response of said patient to drug therapy.

More particularly, said markers are listed in Table 2 and selected from the group comprising SEQ ID NO 1 to 52.

Further, the present invention relates to a method for individualized endocrine therapy, more particularly estrogen receptor therapy, of a patient diagnosed with an endocrine related disease comprising the steps of:

(a) measuring the activity of the estrogen receptor, obtained from a sample from said patient, generating an activity profile, said activity profile comprising the activity of the estrogen receptor from said patient on one or more co-regulator proteins or functional parts thereof in the presence and absence of one or more added phosphatases,

(b) measuring the activity of the estrogen receptor, obtained from a sample from said patient, generating an activity profile, said activity profile comprising the activity of the estrogen receptor from said patient on one or more co-regulator proteins or functional parts thereof in the presence and absence of added estradiol, and,

(c) predicting from said activity profiles the response and optimum dose of said endocrine therapy, more particularly estrogen receptor therapy, to said patient.

Alternatively, the present invention relates to a method for individualized endocrine therapy, more particularly individualized estrogen receptor therapy, of a patient diagnosed with an endocrine related disease comprising the steps of:

(a) measuring the activity of the estrogen receptor, obtained from a sample from said patient, generating an activity profile, said activity profile comprising the activity of the estrogen receptor from said patient on one or more co-regulator proteins or functional parts thereof in the presence and absence of one or more added phosphatases,

(b) measuring the activity of the estrogen receptor, obtained from a sample from said patient, generating an activity profile, said activity profile comprising the activity of the estrogen receptor from said patient on one or more co-regulator proteins or functional parts thereof in the presence and absence of added estradiol,

(c) measuring the activity of the estrogen receptor, obtained from a sample from said patient, generating an activity profile, said activity profile comprising the activity of the estrogen receptor from said patient on one or more co-regulator proteins or functional parts thereof in the presence and absence of added endocrine therapy drug, more particularly estrogen receptor therapy drug, and,

(d) predicting from said activity profiles the response and optimum dose of said endocrine therapy, more particularly estrogen receptor therapy, to said patient.

More particularly, the method for individualized endocrine therapy according to the present invention further comprises a step of measuring the activity of the estrogen receptor, obtained from a sample from said patient, generating an activity profile, said activity profile comprising the activity of the estrogen receptor from said patient on one or more co-regulator proteins or functional parts thereof in the presence and absence of added estradiol and one or more added phosphatases.

More particularly, said one or more co-regulator proteins or functional parts thereof are immobilized onto a solid support.

Further, the present invention relates to a method for individualized endocrine therapy of a patient diagnosed with an endocrine related disorder comprising the steps of:

(a) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of one or more added phosphatases,

(b) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of added ligand of said nuclear receptor, and,

(c) predicting from said activity profiles the response and optimum dose of said endocrine therapy, more particularly estrogen receptor therapy, to said patient.

More particularly, the method for individualized endocrine therapy according to the present invention further comprises a step (c) measuring the binding of the nuclear receptor, obtained from a sample from said endocrine disease from said patient, generating a binding activity profile, said binding activity profile comprising the binding of the nuclear receptor from said patient on one or more immobilized co-regulator proteins or functional parts thereof in the presence and absence of added estradiol and one or more added phosphatases, followed by a step (d) of predicting from the binding profiles the response of said patient to endocrine drug therapy.

More particularly, said one or more co-regulator proteins or functional parts thereof are immobilized onto a solid support.

Further, the present invention relates to a method for individualized endocrine therapy of a patient diagnosed with an endocrine related disorder comprising the steps of:

(a) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of one or more added phosphatases,

(b) measuring the activity of the nuclear receptor, obtained from a sample from said endocrine disease from said patient, generating an activity profile, said activity profile comprising the activity of the nuclear receptor from said patient on one or more co-regulator proteins or functional parts thereof in the presence and absence of added ligand of said nuclear receptor,

(c) measuring the activity of the nuclear receptor, obtained from a sample from said endocrine related disorder from said patient, generating an activity profile, said activity profile comprising the activity of the nuclear hormone receptor from said patient on one or more co-regulator proteins or functional parts thereof in the presence and absence of added endocrine therapy drug, and,

(d) predicting from said activity profiles the response and optimum dose of said endocrine therapy to said patient.

More particularly, the method for individualized endocrine therapy according to the present invention further comprises a step of measuring the binding of the nuclear receptor, obtained from a sample from said endocrine disease from said patient, generating a binding activity profile, said binding activity profile comprising the binding of the nuclear receptor from said patient on one or more immobilized co-regulator proteins or functional parts thereof in the presence and absence of added estradiol and one or more added phosphatases.

Preferably said nuclear receptor consists of the androgen receptor, constitutive androstane receptor, estrogen receptor, farnesoid X receptor, glucocorticoid receptor, liver X receptor, peroxisome proliferator-activated receptor, progesteron receptor, retinoic acid receptor, thyroid receptor or vitamin D3 receptor.

The present invention also relates to an array for carrying out the methods as defined above said array comprising immobilized proteins, peptides or peptide mimetics comprising estrogen receptor binding sites present in at least two peptide markers as listed in Table 1 selected from the group comprising SEQ ID NO 1 to 154 and in particular at least two peptide markers as listed in Table 2 selected from the group comprising SEQ ID NO 1 to 52.

The present invention further relates to a kit for predicting the response of a patient diagnosed with breast cancer to drug treatment, comprising at least one array as defined.

Further, the present invention relates to a method, array or kit according to any of the previous claims allowing the determination of the basal activity levels of the nuclear receptor for use in calibration or normalization or in patient specific calibration or normalization (intra-assay).

In a particular embodiment, the present invention relates to the use of an array comprising a multitude of different immobilized proteins, peptides or peptide mimetics comprising estrogen receptor binding sites present in at least two peptide markers as listed in Table 1 selected from the group comprising SEQ ID NO 1 to 154 for carrying out the methods according to the present invention, and in particular at least two peptide markers as listed in Table 2 selected from the group comprising SEQ ID NO 1 to 52.

Further, the present invention relates to a method, array or kit according to any of the previous embodiments for testing the relevance of the involvement of phosphorylation in the functional properties of a nuclear receptor comprising the steps of any of the above claimed methods.

Further, the present invention relates to the use of a method, array or kit according to any of the previous embodiments to test compounds in patient derived samples for identification of new drugs.

Further the present invention relates to the use of a method, array or kit according to any of the previous embodiments to identify which nuclear receptor cofactor interaction is relevant and subsequent use thereof as a biomarker for screening and identifying more targeted drugs.

Further the present invention relates to a method according to any of the previous embodiments wherein instead of a phosphatase another enzyme is used which is able to remove post-translational modification of the nuclear receptor, such as for instance acetylation, fatty acid modification, sulforylation and methylation.

The nuclear receptor binding sites or peptides usually have a length ranging between 6 and 35 amino acids. A very suitable peptide length ranges between 10 and 30 amino acids. A typical peptide length is 25 amino acids. Each peptide arrayed within the array of co-regulators has a unique sequence.

Co-regulators may be either co-activators or co-repressors. Recently, a number of co-regulatory proteins for nuclear receptors have been identified, and have been shown to act either as co-activators or as co-repressors (reviewed in Horwitz et al., 1996; Shibata et al., 1997; Glass et al., 1997). Among the members of a growing family of co-activators are CBP and members of the SRC-1 gene family including SRC-1/p160 (Onate et al., 1995, Science 270:1354-1357; Halachmi et al., 1994, Science 264:1455-1458; Kamei et al., 1996, Cell 85:403-414), TIF2/GRIP-1 (Voegel et al., 1996, The EMBO Journal 15(14):3667-3675; Hong et al., 1996, Proc. Natl. Acad. Sci. USA 93:4948-4952; Ding et al., 1998, Molecular Endocrinology 12:302-313), and CBP/p300 (Chakravarti et al., 1996, Nature 383:99-103; Hanstein et al., 1996, Proc. Natl. Acad. Sci. USA 93:11540-11545) which function as co-activators of nuclear receptors, and also RIP140 (Cavailles et al., 1994, Proc. Natl. Acad. Sci. USA 91:10009-10013; Cavailles et al., 1995, The EMBO Journal 14(15):3741-3751), TIF1 (Le Douarin et al., 1995 The EMBO Journal 14(9):2020-2033) and TRIP1/SUG-1 (Lee et al., 1995, Nature 374:91-94; vom Baur et al., 1996, The EMBO Journal 15(1):110-124), the functions of which are not clearly defined. Most of these co-regulators of nuclear receptors have a molecular weight around 160 kDa.

Accordingly, in one embodiment of the present invention, a method is provided for measuring compound efficacy and potency on nuclear receptor-co-regulator interaction, wherein said co-regulators are co-activators and/or co-repressors, including fragments thereof, containing a binding domain for the nuclear receptor.

Said binding domain usually comprises a typical residue or an amino acid core consensus. Typical examples of amino acid core consensus sequences are LxxLL, LxxML, FxxFF, and LxxIL (L, leucine; F phenylalanine; M, Methionine; I, isoleucine and x, any amino acid) which is known to be necessary and sufficient to mediate the binding of co-regulator proteins to liganded classical nuclear receptors. In addition, co-regulator motifs other than the above mentioned which may enable interaction with an LBD are equally contemplated within the present invention.

Accordingly, in one embodiment of the present invention, a method is provided for measuring compound efficacy and potency on nuclear receptor-co-regulator interaction, wherein said co-regulators are in the form of peptides comprising the amino acid core consensus sequence chosen from the group comprising LxxLL, LxxML, FxxFF, and LxxIL.

Within the methods of the present invention, the peptide array format may be chosen out of various formats including, but not limited to free peptides in separate vials such as small eppendorf vials with each unique peptide sequence per vial, peptides coupled onto microspheres with each unique peptide sequence onto a separate microsphere, or peptides immobilized onto a solid support in the format of a microarray having each unique peptide sequence coupled onto a distinct spot on the solid surface. Typically within the methods of the present invention, the peptide array format is a microarray.

The expression “immobilized” or “coupled” onto a microsphere, solid support or other carrier as used in the present specification refers to the attachment or adherence of one or more molecules to the surface of the carrier including attachment or adherence to the inner surface of said carrier in the case of e.g. a porous or flow-through solid support.

A number of materials suitable for use as a microarray solid support in the present invention have been described in the art. Materials particularly suitable for use as microarray solid support in the present invention include any type of solid support, including solid supports, known in the art, e.g. glass microscope slides, silicon chips or nylon membranes.

Particular suitable microarray solid supports for use within the methods of the present invention are porous supports. The term “porous support” as used in the present specification refers to a support possessing or full of pores, wherein the term “pore” refers to a minute opening or microchannel by which matter may be either absorbed or passed through. Particularly, where the pores allow passing-through of matter, the support is likely to be permeable.

Particular useful porous supports for employment within the methods described in the present specification are 3-dimensional supports, which allow pressurized movement of fluid up and down (i.e., cycling) through the pores, e.g. the sample solution, through its structure.

As such, particular useful porous supports for use within the present methods possess a flow-through nature. The channels or pores through a flow-through solid support may be discrete or branched having extremities typically ending at the corresponding top and bottom surface of the solid support. In contrast with two-dimensional supports, 3-dimensional microarray supports suitable within the methods as described herein give significantly reduced hybridization times and increased signal and signal-to-noise ratios.

Accordingly, in one embodiment of the present invention, a method is provided wherein said microarray is a flow-through microarray.

Suitable 3-dimensional solid supports for use within the present invention may be manufactured out of, for example, a metal, a ceramic metal oxide or an organic polymer. In view of strength and rigidity, a metal or a ceramic metal oxide may be used. Above all, in view of heat resistance and chemicals resistance, a metal oxide may be used. In addition, metal oxides provide a support having both a high channel density and a high porosity, allowing high density arrays comprising different first binding substances per unit of the surface for sample application. In addition, metal oxides are highly transparent for visible light. Metal oxides are relatively cheap that do not require the use of any typical microfabrication technology and, that offers an improved control over the liquid distribution over the surface of the support, such as an electrochemically manufactured metal oxide membrane.

Typically, flow-through microarray solid supports such as metal oxide solid supports may undergo positive and negative pressures. By applying alternative positive and negative pressure to the arrays a sample solution may be dynamically pumped up and down through the support pores. Said dynamical pumping allows immediate real-time detection of generated products from a reaction which takes place within the pores of the support. The expression “positive pressure” relates to a pressure higher than the standard atmospheric pressure of 1 atm. The expression “negative pressure” relates to a pressure lower than the standard atmospheric pressure of 1 atm. A negative pressure is also referred to as vacuum pressure.

Metal oxide supports or membranes suitable for use within the methods of the present invention may be anodic oxide films. WO 99/02266 which discloses the Anopore™ porous membrane or support is exemplary in this respect, and is specifically incorporated by reference in the present invention.

As well known in the art, aluminum metal may be anodized in an electrolyte to produce an anodic oxide film. The anodization process results in a system of larger pores extending from one face, e.g., the top surface of a solid support, and interconnects with a system of smaller pores extending from the other face or bottom surface. Pore size is determined by the minimum diameters of the smaller pores, while flow rates are determined largely by the length of the smaller pores, which can be made very short. Accordingly, such membranes may have oriented through-going partially branched channels with well-controlled diameter and useful chemical surface properties. The expression “partially branched” as used within the present description refers to larger channels as being branched at one end into a series of smaller channels. The larger channels which predominantly run in parallel are usually mutually interconnected, resulting in so-called substantially discrete channels, and a similar interconnection may appear between the smaller channels.

Useful thickness of solid supports or membranes suitable for use within the methods of the present invention may for instance range from 30 μm to 150 μm (including thicknesses of 30 40, 50, 60, 70, 80, 90, 100, 110, 120, 130, 140 and 150 μm). A particular suitable example of support thickness is 60 μm.

A suitable support pore diameter for porous solid supports, in particular flow-through solid supports, ranges from 150 to 250 nm including 150, 160, 170, 180, 190, 200, 210, 220, 230, 240 and 250 nm. A particular suitable example of pore diameter is 200 nm.

Within the methods of the present invention, co-regulators are arrayed, typically in a microarray format comprising various spots with each spot having immobilized thereto a unique peptide sequence representing the typical LxxLL, LxxML, FxxFF, LxxIL or other binding region of a co-activator or a co-repressor. Typically, a microarray comprises tens to hundreds spots or small spatial areas on the solid surface. Within the methods of the present invention the number of spots on a microarray ranges between 50 and 1000 spots. A very suitable number of spots range between 150 and 700 spots. A typical number of spots on a microarray for use within the methods of the present invention is 400, corresponding to a spot density of about 25 spots per mm².

The number of spots on a microarray suitable for use within the present methods may accommodate the immobilization of various different peptides as well as various different peptide concentrations.

Accordingly, within the methods of the present invention said nuclear receptor family comprises receptors for glucocorticoids (GRs), androgens (ARs), mineral corticoids (MRs), progestins (PRs), estrogens (ERs), thyroid hormones (TRs), vitamin D (VDRs), retinoids (RARs and RXRs), steroids, peroxisomes (XPARs and PPARs), oxysterols (LXRs), bile acids (FXRs), and icosanoids (IRs). The so-called “orphan receptors” for which ligands have not been identified are also part of the nuclear receptor super family, as they are structurally homologous to the classic nuclear receptors, such as steroid and thyroid receptors.

The detection signal can be in the form of a fluorescent signal, chemiluminescent signal, or a calorimetric signal. A particular useful detector system in the methods as described herein includes labeling of the NR to provide a detection system which may generate a detectable signal which is indicative of the interaction of an analyte with an immobilized target. The detectable label may be a direct detectable label for instance a fluorescent label on the NR or may be an indirect label using for instance an antibody against an epitope on the NR which does not influence the binding reaction between the NR and the NR box. This antibody may be labeled directly with for instance a fluorescent label or indirectly using a secondary antibody with a detectable label for instance a fluorescent label.

The term “label” as used in this specification refers to a molecule propagating a signal to aid in detection and quantification. Said signal may be detected either visually (e.g., because it has color, or generates a color product, or emits fluorescence) or by use of a detector that detects properties of the reporter molecule (e.g., radioactivity, magnetic field, etc.). In the present specification, labels allow for the detection of the interaction between NR and co-regulator sequence. Detectable labels suitable for use in the present invention include but are not limited to any composition detectable by spectroscopic, photochemical, biochemical, immunochemical, electrical, optical or chemical means. Where appropriate, the system may contain more labels producing different signals which may be a component of, or released by, an interaction event. Any combination of labels, e.g. first and second labels, first, second, and third labels, etc. may be employed for analyte sets, provided the labels are distinguishable from one another. Examples of distinguishable labels are well-known in the art and include: two or more different wavelength fluorescent dyes, such as Cy3 and Cy5 or Alexa 488, Alexa 542 and Bodipy 630/650; two or more isotopes with different energy of emission, such as ³²P and ³³P; labels which generate signals under different treatment conditions, like temperature, pH treatment by additional chemical agents, etc.; and labels which generate signals at different time points after treatment.

Particular suitable labels that may be employed in the present invention may be chromogens including those that absorb light in a distinctive range of wavelengths so that a color may be observed or, alternatively, that emit light when irradiated with radiation of a particular wavelength or wavelength range, e.g., fluorescent molecules. Particular useful fluorescent labels include, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa 488, Alexa542, etc), green fluorescent protein (GFP), blue fluorescent protein (BFP), yellow fluorescent protein (YFP), red fluorescent protein (RFP), and the like, (see, e.g., Molecular Probes, Eugene, Oreg., USA).

The detection of a signal profile allows determination of the specificity of an NR-co-regulator interaction modulated by a compound. The modulation of the interaction activity and/or specificity may be deduced from a comparison of the signal profile with a signal profile drawn up in the presence of increasing or decreasing co-regulator concentrations, including the absence of co-regulator.

The following examples are offered by way of illustration, and not by way of limitation.

### EXAMPLES

### Example 1

Study the effect of post-translational modifications on the estrogen receptor alpha in clinical samples on response to estradiol and tamoxifen.

Post-translational modifications (PTM) on the Estrogen Receptor alpha (ERα) and other nuclear receptors have been shown to influence coregulator binding. Phosphorylation of ERαSer305, induced by protein kinase A (PKA), has been linked to resistance to tamoxifen treatment. Under tamoxifen, a known antagonist of the ERα, this phosphorylation affects the conformation of ERα and changes its orientation to the protein SRC-1. In this example we studied the effect of ERαSer305 phosphorylation on the binding of cofactors in transfected cells and in breast tumor specimens. We used the methods and coregulator peptide array as described in WO 2008/028978 containing 154 coregulator peptides (table 1).

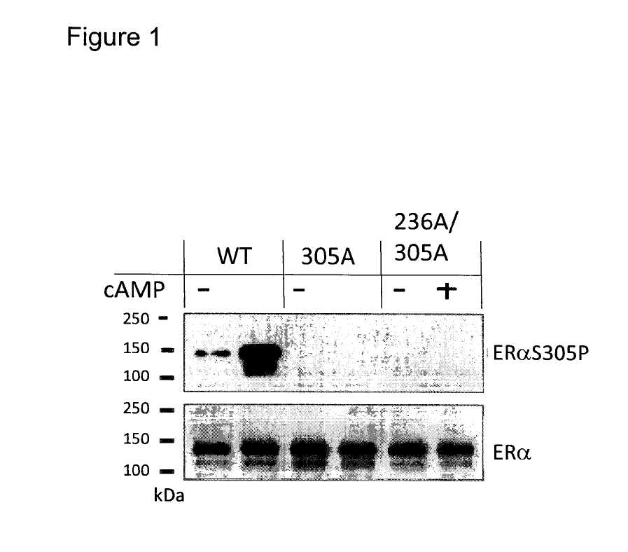

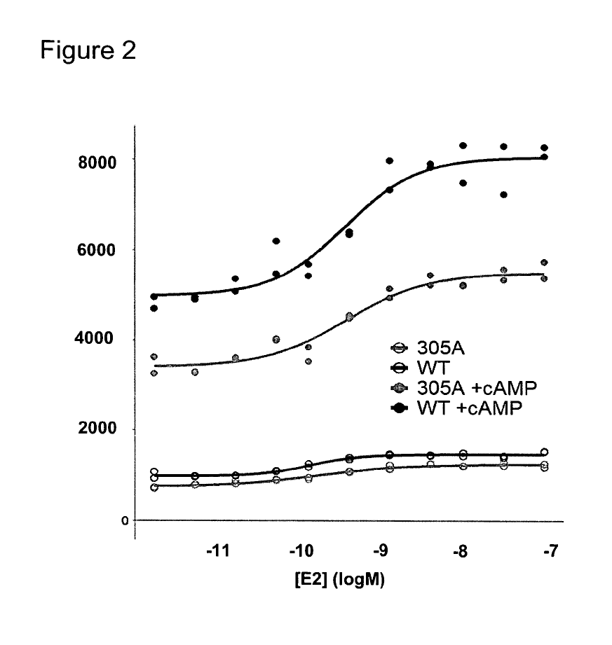

ERαY/C-transfected U2OS cells were stimulated with cAMP to induce PKA-mediated ERα phosphorylation. The serine-to-alanine mutant ERαSer305Ala-Y/C was used as a negative control (FIG. 1). Next, we incubated the lysates with a concentration range of estradiol (E2) up to 10⁻⁸ M monitored the effect on ERα binding to the coregulator derived peptides on the microarray. This resulted in a dose dependent modulation of ERα binding to coregulators, as illustrated by the control peptide (IDNR13) in FIG. 2 using the method according to WO 2008/028978. Wild type and mutant ERα from cAMP stimulated cells and non-stimulated control cells were responsive to estradiol.

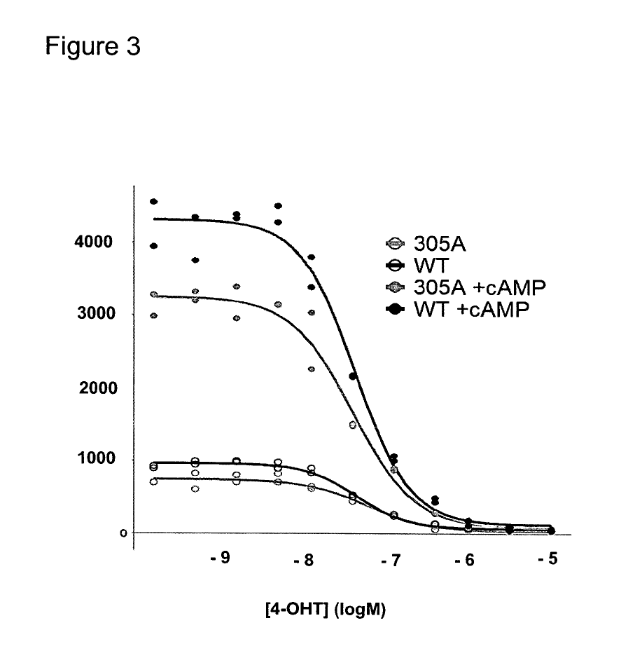

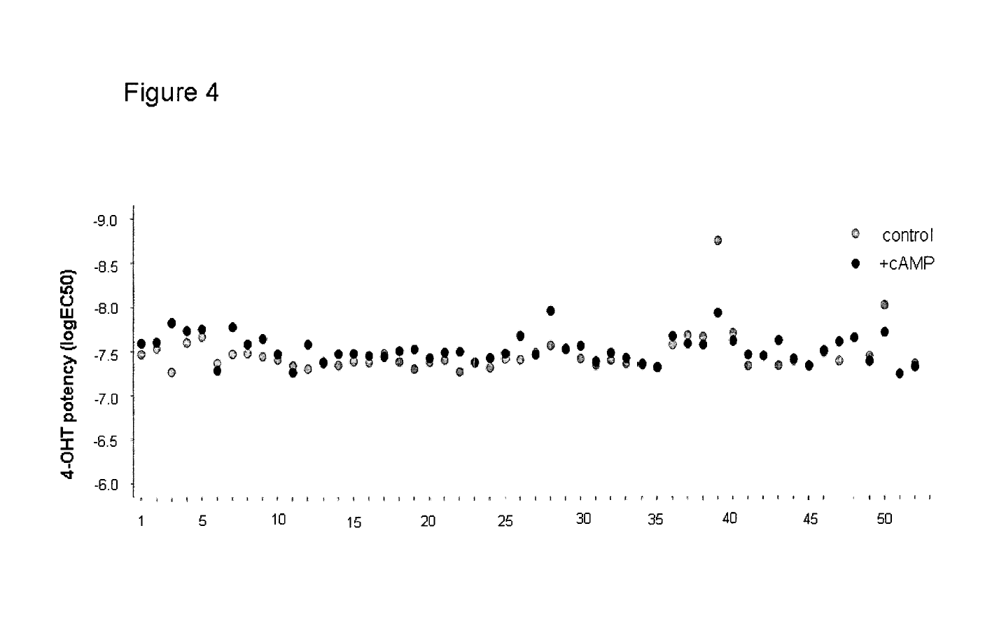

The effect of PKA activation on peptide binding was also studied under tamoxifen conditions. This is illustrated by ERα binding to the control peptide (IDNR13) using a concentration range of tamoxifen (4-OH) up to 10-5 M (FIG. 3) showing a largely blocked estrogen receptor at saturating tamoxifen concentrations. Although upon PKA activation the initial binding (LIB) of ERα-wt was higher than of ERαSer305Ala, binding of both was largely blocked at saturating tamoxifen concentrations. The potency (EC50) of 4OH-tamoxifen was not affected and largely similar for the various motifs on the chip (FIG. 4) which shows that enhanced ERα activity by phosphorylation, leads to ligand independent activation and/or enhanced response to E2, which results in enhanced residual receptor activity at non-saturating tamoxifen levels.

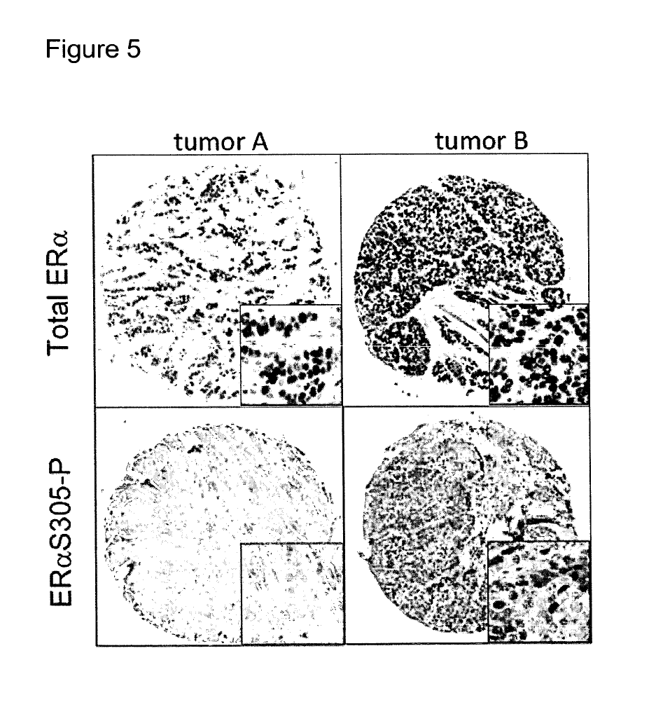

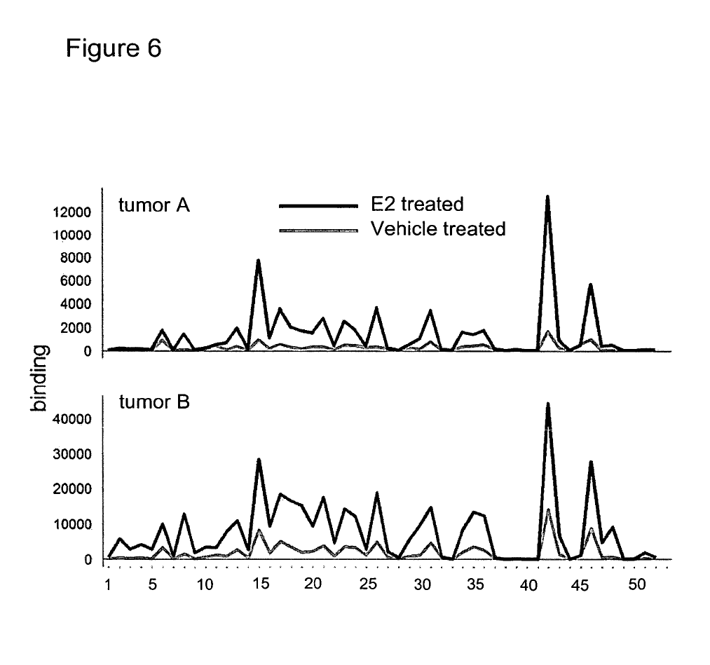

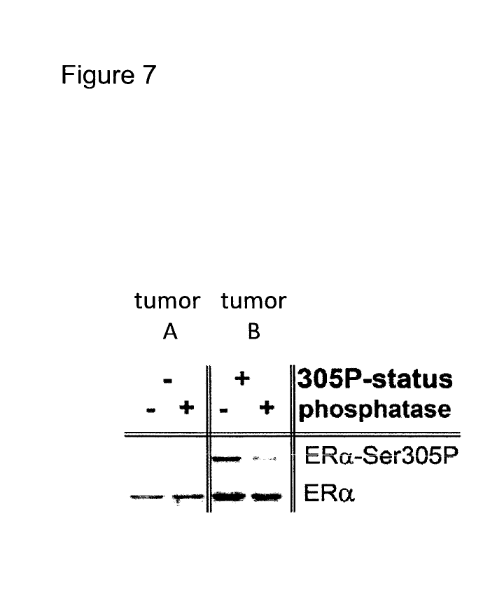

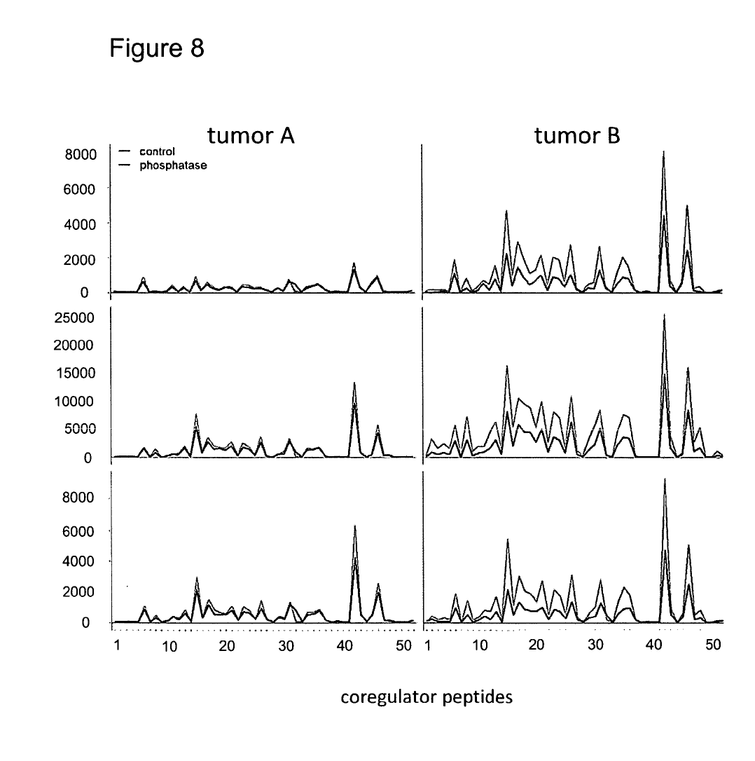

Two breast tumors were profiled using immunohistochemical evaluation. Both tumors stained positive for ERα and one was negative (tumor A) and one was positive (tumor B) for phosphorylated ERα Ser305, as shown in Western blot (FIG. 5). These ERα+ tumors are still responsive to estradiol (E2) compared to DMSO vehicle treatment, showing increased peptide binding on the array according to the method as outlined WO 2008/028978 (FIG. 6). The role of S305 phosphorylation on receptor activity was assessed by dephosphorylation of the receptor in the lysates by addition of lambda phosphatase. This largely reduced the level of receptor phosphorylation in the ERαSer305-P positive tumor (FIG. 7). ERα showed enhanced binding in the ERαSer305-P positive tumor, implying a more active receptor. Phosphatase treatment strongly reduced binding of ERα from the Ser305-P positive tumor B (FIG. 8, right panel) to coregulators, while the binding levels of the unphosphorylated receptor from tumor A (left panel) were unaffected. Dephosphorylation of ERα in tumor B reduces ligand independent activity vehicle (control) as well as the response to estradiol (E2) and the residual activity after tamoxifen (Tam) (EC50) treatment.

TABLE 1
List of List of coregulator-derived sequences each containing an
LxxLL domain (NR box) used in the method according to WO 2008/028978.
ID as follows: [coregulator]_[aa start]_[aa end of peptide]

| SEQ ID | | |
|---|---|---|
| NO | Name | Sequence |
| 1 | BL1S1_1_11 | MLSRLLKEHQA |
| 2 | BRD8_254_276 | TVAASPAASGAPTLSRLLEAGPT |
| 3 | CBP_57_80 | GNLVPDAASKHKQLSELLRGGSGS |
| 4 | EP300_69_91 | GMVQDAASKHKQLSELLRSGSSP |
| 5 | HAIR_745_767_C755S/C759S | EDRAGRGPLPSPSLSELLASTAV |
| 6 | IKBB_277_299 | PLGSAMLRPNPILARLLRAHGAP |
| 7 | ILK_131_153 | KYGEMPVDKAKAPLRELLRERAE |
| 8 | JHD2C_2054_2076 | PLVSQNNEQGSTLRDLLTTTAGK |
| 9 | LCOR_40_62 | TTSPTAATTQNPVLSKLLMADQD |
| 10 | MED1_591_614 | HGEDFSKVSQNPILTSLLQITGNG |
| 11 | MLL2_4175_4197 | LLAGPRSEAGHLLLQKLLRAKNV |
| 12 | NCOA1_620_643 | SDGDSKYSQTSHKLVQLLTTTAEQ |
| 13 | NCOA1_677_700 | PSSHSSLTERHKILHRLLQEGSPS |
| 14 | NCOA1_737_759 | ASKKKESKDHQLLRYLLDKDEKD |
| 15 | NCOA1_1421_1441 | TSGPQTPQAQQKSLLQQLLTE |
| 16 | NCOA2_628_651 | GQSRLHDSKGQTKLLQLLTTKSDQ |
| 17 | NCOA2_677_700 | STHGTSLKEKHKILHRLLQDSSSP |
| 18 | NCOA2_733_755 | EPVSPKKKENALLRYLLDKDDTK |
| 19 | NCOA3_609_631 | QRGPLESKGHKKLLQLLTCSSDD |
| 20 | NCOA3_673_695 | MHGSLLQEKHRILHKLLQNGNSP |
| 21 | NCOA3_725_747 | EQLSPKKKENNALLRYLLDRDDP |
| 22 | NR0B1_1_23 | MAGENHQWQGSILYNMLMSAKQT |
| 23 | NR0B1_68_90_C69S | FSGKDHPRQGSILYSMLTSAKQT |
| 24 | NR0B1_136_159 | GEDHPRQGSILYSLLTSSKQTHVA |
| 25 | NR0B2_9_31_C9S/C11S | SPSQGAASRPAILYALLSSSLKA |
| 26 | NR0B2_106_128 | TFEVAEAPVPSILKKILLEEPSS |
| 27 | NR0B2_201_223_C207S | EVLEPWSPAAQGRLTRVLLTAST |
| 28 | NRBF2_128_150 | PEIQGIFDRDPDTLLYLLQQKSE |
| 29 | NRIP1_120_142 | VDSVPKGKQDSTLLASLLQSFSS |
| 30 | NRIP1_253_275_C263S | PATSPKPSVASSQLALLLSSEAH |
| 31 | NRIP1_368_390 | RNNIKQAANNSLLLHLLKSQTIP |
| 32 | NRIP1_488_510 | KNSKLNSHQKVTLLQLLLGHKNE |
| 32 | NRIP1_488_510 | KNSKLNSHQKVTLLQLLLGHKNE |
| 33 | NRIP1_805_831 | PVSPQDFSFSKNGLLSRLLRQNQDSYL |
| 34 | NRIP1_924_946 | RSWARESKSFNVLKQLLLSENCV |
| 35 | NRIP1_1055_1077 | EKDSPRLTKTNPILYYMLQKGGN |
| 36 | NSD1_894_916 | SSQNHIPIEPDYKFSTLLMMLKD |
| 37 | PELP1_20_42 | GTGGLSAVSSGPRLRLLLLESVS |
| 38 | PELP1_446_468 | AGMLQGGASGEALLTHLLSDISP |
| 39 | PNRC1_306_327 | ENSNQNRELMAVHLKTLLKVQT |
| 40 | PPRC1_151_173 | DSELLVSPREGSSLHKLLTLSRT |
| 41 | PRGC1_130_155 | DGTPPPQEAEEPSLLKKLLLAPANTQ |
| 42 | PRGC2_146_166 | PAPEVDELSLLQKLLLATSYP |
| 43 | PRGC2_338_358 | AEFSILRELLAQDVLCDVSKP |
| 44 | PROX1_57_79 | SVVQHADGEKSNVLRKLLKRANS |
| 45 | TIF1A_747_769 | ESRPQNANYPRSILTSLLLNSSQ |
| 46 | TIP60_476_498 | LSEDIVDGHERAMLKRLLRIDSK |
| 47 | TREF1_168_190 | TQSAVMDGAPDSALRQLLSQKPM |
| 48 | TREF1_850_872 | HSLFEAKGDVMVALEMLLLRKPV |
| 49 | TRXR1_132_154 | GHGPTLKAYQEGRLQKLLKMNGP |
| 50 | WIPI1_119_141 | ESIYIHNIKDMKLLKTLLDIPAN |
| 51 | WIPI1_313_335_C318S | GQRNISTLSTIQKLPRLLVASSS |
| 52 | ZNHI3_89_111 | LQNLKNLGESATLRSLLLNPHLR |
| 53 | ANDR_10_32 | VYPRPPSKTYRGAFQNLFQSVRE |
| 54 | CBP_2055_2077 | SVQPPRSISPSALQDLLRTLKSP |
| 55 | CBP_345_367_C367S | TADPEKRKLIQQQLVLLLHAHKS |
| 56 | CBP_345_368 | TADPEKRKLIQQQLVLLLHAHKCQ |
| 57 | CBP_345_368_C367S | TADPEKRKLIQQQLVLLLHAHKSQ |
| 58 | CCND1_243_264_C243S/C247S | SLRASQEQIEALLESSLRQAQQ |
| 59 | CENPR_1_18 | MPVKRSLKLDGLLEENSF |
| 60 | CENPR_159_177 | PHKASRHLDSYEFLKAILN |
| 61 | CHD9_1023_1045 | LLTGTPLQNTVEELFSLLHFLEP |
| 62 | CHD9_2018_2040 | QYQVALSASPLTSLPRLLDAKGI |
| 63 | CHD9_855_877 | KNGNQLREYQLEGLNWLLFNWYN |
| 64 | CNOT1_140_162 | DLRGFAAQFIKQKLPDLLRSYID |
| 65 | CNOT1_1626_1648 | IPPTLAMNPQAQALRSLLEVVVL |
| 66 | CNOT1_1929_1951_C1932S | RAKSYHNLDAFVRLIALLVKHSG |
| 67 | CNOT1_2083_2105 | ELTKPMQILYKGTLRVLLVLLHD |
| 68 | CNOT1_2086_2108 | KPMQILYKGTLRVLLVLLHDFPE |
| 69 | CNOT1_557_579 | LSRILDVAQDLKALSMLLNGTPF |
| 70 | DDX5_133_155 | DMVGVAQTGSGKTLSYLLPAIVH |
| 71 | DHX30_241_262 | QFPLPKNLLAKVIQIATSSSTA |
| 72 | DHX30_49_70 | EFPQPKNLLNSVIGRALGISHA |
| 73 | EP300_2039_2061 | SPLKPGTVSQQALQNLLRTLRSP |
| 74 | GELS_376_398 | QVSVLPEGGETPLFKQFFKNWRD |
| 75 | GNAQ_21_43 | RQLRRDKRDARRELKLLLLGTGE |
| 76 | HAIR_553_575_C567S | GLAKHLLSGLGDRLSRLLRRERE |
| 77 | IKBB_244_266 | PLHLAVEAQAADVLELLLRAGAN |
| 78 | IKBB_62_84 | LHLAVIHQHEPFLDFLLGFSAGT |
| 79 | KIF11_832_854_C854S | QWVSSLNEREQELHNLLEVVSQS |
| 80 | L3R2A_12_34 | WHNNVLHTHLVFFLPHLLNQPFS |
| 81 | MAPE_249_271 | LAKFSPYLGQMINLRRLLLSHIH |
| 82 | MAPE_300_322 | ALYVDSLFFLRGRLDQLLRHVMN |
| 83 | MAPE_356_378 | LSGVMLTDVSPEPLQALLERASA |
| 84 | MAPE_382_404_C388S | DLVFDESGITDDQLLALLPSLSH |
| 85 | MAPE_454_476_C472S | TLHLERLAYLHARLRELLSELGR |
| 86 | MAPE_91_113 | HLHLETFKAVLDGLDVLLAQEVR |
| 87 | MED1_632_655 | VSSMAGNTKNHPMLMNLLKDNPAQ |
| 88 | MEN1_255_277 | HTDSLELLQLQQKLLWLLYDLGH |
| 89 | MGMT_86_108 | HPVFQQESFTRQVLWKLLKVVKF |
| 90 | MLL2_4702_4724 | PRLKKWKGVRWKRLRLLLTIQKG |
| 91 | MTA1S_388_410_C393S/C396S | GAGRASESSYMSSLRILLDILEE |
| 92 | NCOA2_866_888 | SQSTFNNPRPGQLGRLLPNQNLP |
| 93 | NCOA3_104_123_N-KKK | KKKGQGVIDKDSLGPLLLQALDG |
| 94 | NCOA3_609_631_C627S | QRGPLESKGHKKLLQLLTSSSDD |
| 95 | NCOA3_MOUSE_1029_1051 | HGSQNRPLLRNSLDDLLGPPSNA |
| 96 | NCOA4_315_337 | RKPENGSRETSEKFKLLFQSYNV |
| 97 | NCOA4_79_101_C101S | QLKEETLQQQAQQLYSLLGQFNS |
| 98 | NCOA6_1479_1501 | LVSPAMREAPTSLSQLLDNSGAP |
| 99 | NCOA6_875_897 | PVNKDVTLTSPLLVNLLQSDISA |
| 100 | NCOR1_1925_1946 | TTITAANFIDVIITRQIASDKD |
| 101 | NCOR1_2039_2061 | MGQVPRTHRLITLADHICQIITQ |
| 102 | NCOR1_2039_2061_C2056S | MGQVPRTHRLITLADHISQIITQ |
| 103 | NCOR1_2251_2273 | GHSFADPASNLGLEDIIRKALMG |
| 104 | NCOR1_2376 2398 | SSTGSTQFPYNPLTMRMLSSTPP |
| 105 | NCOR1_662_684_C662S | SKNFYFNYKRRHNLDNLLQQHKQ |
| 106 | NCOR2_2123_2145 | APGVKGHQRVVTLAQHISEVITQ |
| 107 | NCOR2_2330_2352 | QAVQEHASTNMGLEAIIRKALMG |
| 108 | NCOR2_649_671_C649S | SKNFYFNYKKRQNLDEILQQHKL |
| 109 | NELFB_328_350 | QETLPRDSPDLLLLLRLLALGQG |
| 110 | NELFB_428_450 | YVLHITKQRNKNALLRLLPGLVE |
| 111 | NELFB_80_102 | IASEGKAEERYKKLEDLLEKSFS |
| 112 | NR0B2_237_257 | FRPIIGDVDIAGLLGDMLLLR |
| 113 | NRIP1_121_143_P124R | DSVRKGKQDSTLLASLLQSFSSR |
| 114 | NRIP1_173_195 | KDLRCYGVASSHLKTLLKKSKVK |
| 115 | NRIP1_173_195_C177S | KDLRSYGVASSHLKTLLKKSKVK |
| 116 | NRIP1_700_722 | GSEIENLLERRTVLQLLLGNPNK |
| 117 | NRIP1_701_723 | SEIENLLERRTVLQLLLGNPTKG |
| 118 | NRIP1_8_30 | GSDVHQDSIVLTYLEGLLMHQAA |
| 119 | NRIP1_924_946_C945S | RSWARESKSFNVLKQLLLSENSV |
| 120 | NSD1_982_1004 | GGDSALSGELSASLPGLLSDKRD |
| 121 | PAK6_248_270 | SPKTRESSLKRRLFRSMFLSTAA |
| 122 | PCAF_178_200 | EEDADTKQVYFYLFKLLRKSILQ |
| 123 | PELP1_142_164 | QDPPATMELAVAVLRDLLRYAAQ |
| 124 | PELP1_168_190 | LFRDISMNHLPGLLTSLLGLRPE |
| 125 | PELP1_251_273 | SQGLKHTESWEQELHSLLASLHT |
| 126 | PELP1_258_280 | ESWEQELHSLLASLHTLLGALYE |
| 127 | PELP1_496_518_C496S | SPFFLQSLHGDGPLRLLLLPSIH |
| 128 | PELP1_56_78_C71S | VHPPNRSAPHLPGLMSLLRLHGS |
| 129 | PELP1_571_593_C575S/C581S | TSSRSRRELYSLLLALLLAPSPR |
| 130 | PIAS2_6_28 | ELRNMVSSFRVSELQVLLGFAGR |
| 131 | PNRC2_118_139 | SFNPSDKEIMTFQLKTLLKVQV |
| 132 | PPRC1_1159_1181 | QAFISEIGIEASDLSSLLEQFEK |
| 133 | PR285_1062_1084 | WPWDGELNADDAILRELLDESQK |
| 134 | PR285_1105_1127 | QQARLYENLPPAALRKLLRAEPE |
| 135 | PR285_1160_1182_C1163S | RLDSGMAFAGDEVLVQLLSGDKA |
| 136 | PR285_2216_2238_C2219S | ILYSGPSNKSVDVLAGLLLRRME |
| 137 | PR285_432_454_C453S/C454S | EVRLERRASSGQALWLLLPARSS |
| 138 | PRDM2_948_970 | SPALQTPSLSSGQLPPLLIPTDP |
| 139 | PRGC1_134_154 | PPQEAEEPSLLKKLLLAPANT |
| 140 | PRGR_102_124 | SPPEKDSGLLDSVLDTLLAPSGP |
| 141 | PRGR_42_64_C64S | SDTLPEVSAIPISLDGLLFPRPS |
| 142 | RAD9A_348_370 | EPSTVPGTPPPKKFRSLFFGSIL |
| 143 | RBL2_875_897_C879S/C894S | SIIQSPELMMDRHLDQLLMSAIY |
| 144 | TF65_437_459 | SEALLQLQFDDEDLGALLGNSTD |
| 145 | TGFI1_325_347_C334S/C346S | FHEREGRPYSRRDFLQLFAPRSQ |
| 146 | TGFI1_443_461_C452S/C455S | FQERAGKPYSQPSFLKLFG |
| 147 | TIF1A_373_395_C394S | TALLYSKRLITYRLRHLLRARSD |
| 148 | TRIP4_149_171_C171S | FVNLYTRERQDRLAVLLPGRHPS |
| 149 | TRRAP_3535_3557_C3535S/C3555S | SLTESRREERVLQLLRLLNPSLE |
| 150 | TRRAP_770_792 | GSHDLLYQEFLPLLPNLLQGLNM |
| 151 | TRRAP_971_993 | LVAMMSLEDNKHALYQLLAHPNF |
| 152 | UBE3A_396_418 | DDEEPIPESSELTLQELLGEERR |
| 153 | UBE3A_649_671 | RDLGDSHPVLYQSLKDLLEYEGN |
| 154 | ZNT9_449_471 | LLGRSIQPEQVQRLTELLENDPS |

TABLE 2
List of selected 52 coregulator-derived sequences each containing an
LxxLL domain (NR box) used in the method according to WO 2008/028978.
ID as follows: [coregulator]_[aa start]_[aa end of peptide]

| SEQ ID NO | Name | Sequence |
|---|---|---|
| 1 | BL1S1_1_11 | MLSRLLKEHQA |
| 2 | BRD8_254_276 | TVAASPAASGAPTLSRLLEAGPT |
| 3 | CBP_57_80 | GNLVPDAASKHKQLSELLRGGSGS |
| 4 | EP300_69_91 | GMVQDAASKHKQLSELLRSGSSP |
| 5 | HAIR_745_767_C755S/C759S | EDRAGRGPLPSPSLSELLASTAV |
| 6 | IKBB_277_299 | PLGSAMLRPNPILARLLRAHGAP |
| 7 | ILK_131_153 | KYGEMPVDKAKAPLRELLRE RAE |
| 8 | JHD2C_2054_2076 | PLVSQNNEQGSTLRDLLTTTAGK |
| 9 | LCOR_40_62 | TTSPTAATTQNPVLSKLLMADQD |
| 10 | MED1_591_614 | HGEDFSKVSQNPILTSLLQITGNG |
| 11 | MLL2_4175_4197 | LLAGPRSEAGHLLLQKLLRAKNV |
| 12 | NCOA1_620_643 | SDGDSKYSQTSHKLVQLLTTTAEQ |
| 13 | NCOA1_677_700 | PSSHSSLTERHKILHRLLQEGSPS |
| 14 | NCOA1_737_759 | ASKKKESKDHQLLRYLLDKDEKD |
| 15 | NCOA1_1421_1441 | TSGPQTPQAQQKSLLQQLLTE |
| 16 | NCOA2_628_651 | GQSRLHDSKGQTKLLQLLTTKSDQ |
| 17 | NCOA2_677_700 | STHGTSLKEKHKILHRLLQDSSSP |
| 18 | NCOA2_733_755 | EPVSPKKKENALLRYLLDKDDTK |
| 19 | NCOA3_609_631 | QRGPLESKGHKKLLQLLTCSSDD |
| 20 | NCOA3_673_695 | MHGSLLQEKHRILHKLLQNGNSP |
| 21 | NCOA3_725_747 | EQLSPKKKENNALLRYLLDRDDP |
| 22 | NR0B1_1_23 | MAGENHQWQGSILYNMLMSAKQT |
| 23 | NR0B1_68_90_C69S | FSGKDHPRQGSILYSMLTSAKQT |
| 24 | NR0B1_136_159 | GEDHPRQGSILYSLLTSSKQTHVA |
| 25 | NR0B2_9_31_C9S/C11S | SPSQGAASRPAILYALLSSSLKA |
| 26 | NR0B2_106_128 | TFEVAEAPVPSILKKILLEEPSS |
| 27 | NR0B2_201_223_C207S | EVLEPWSPAAQGRLTRVLLTAST |
| 28 | NRBF2_128_150 | PEIQGIFDRDPDTLLYLLQQKSE |
| 29 | NRIP1_120_142 | VDSVPKGKQDSTLLASLLQSFSS |
| 30 | NRIP1_253_275_C263S | PATSPKPSVASSQLALLLSSEAH |
| 31 | NRIP1_368_390 | RNNIKQAANNSLLLHLLKSQTIP |
| 32 | NRIP1_488_510 | KNSKLNSHQKVTLLQLLLGHKNE |
| 32 | NRIP1_488_510 | KNSKLNSHQKVTLLQLLLGHKNE |
| 33 | NRIP1_805_831 | PVSPQDFSFSKNGLLSRLLRQNQDSYL |
| 34 | NRIP1_924_946 | RSWARESKSFNVLKQLLLSENCV |
| 35 | NRIP1_1055_1077 | EKDSPRLTKTNPILYYMLQKGGN |
| 36 | NSD1_894_916 | SSQNHIPIEPDYKFSTLLMMLKD |
| 37 | PELP1_20_42 | GTGGLSAVSSGPRLRLLLLESVS |
| 38 | PELP1_446_468 | AGMLQGGASGEALLTHLLSDISP |
| 39 | PNRC1_306_327 | ENSNQNRELMAVHLKTLLKVQT |
| 40 | PPRC1_151_173 | DSELLVSPREGSSLHKLLTLSRT |
| 41 | PRGC1_130_155 | DGTPPPQEAEEPSLLKKLLLAPANTQ |
| 42 | PRGC2_146_166 | PAPEVDELSLLQKLLLATSYP |
| 43 | PRGC2_338_358 | AEFSILRELLAQDVLCDVSKP |
| 44 | PROX1_57_79 | SVVQHADGEKSNVLRKLLKRANS |
| 45 | TIF1A_747_769 | ESRPQNANYPRSILTSLLLNSSQ |
| 46 | TIP60_476_498 | LSEDIVDGHERAMLKRLLRIDSK |
| 47 | TREF1_168_190 | TQSAVMDGAPDSALRQLLSQKPM |
| 48 | TREF1_850_872 | HSLFEAKGDVMVALEMLLLRKPV |
| 49 | TRXR1_132_154 | GHGPTLKAYQEGRLQKLLKMNGP |
| 50 | WIPI1_119_141 | ESIYIHNIKDMKLLKTLLDIPAN |
| 51 | WIPI1_313_335_C318S | GQRNISTLSTIQKLPRLLVASSS |
| 52 | ZNHI3_89_111 | LQNLKNLGESATLRSLLLNPHLR |

1. A method for predicting the response of a patient diagnosed with breast cancer to treatment with an estrogen receptor therapy drug, comprising the steps of:
(a) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of one or more added phosphatases,
(b) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol,
(c) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol and one or more added phosphatases, and,
(d) predicting from said binding profiles the response of said patient to estrogen receptor drug therapy.

2. Method according to claim 1 wherein between step (a) and (d) an additional step of measuring on the basis of a sample the binding of the estrogen receptor, obtained from a breast cancer tumor from said patient, generating a binding profiling, said binding profile comprising the binding of the estrogen receptor from said patient on one or more immobilized co-regulator proteins or functional parts thereof in the presence and absence of one or more added drugs is included.

3. The method according to claim 1 or 2 wherein said estrogen receptor therapy drug consists of or comprises tamoxifen, raloxifen or aromatase inhibitors.

4. The method according to any of claims 1 to 3, wherein said binding profiling are determined on immobilized proteins, peptides or peptide mimetics immobilized on a solid support, and preferably immobilized on a porous solid support.

5. The method according to claim 4, wherein said immobilized peptides are at least two peptides as listed in Table 1 selected from a group consisting of any of the SEQ ID NO 1 to 154.

6. The method according to claim 5, wherein said immobilized peptides are at least SEQ ID NO 1 to 52 as listed in Table 2.

7. A method for predicting the response of a patient diagnosed with breast cancer to drug treatment according to any of the previous claims, comprising the steps of:
(a) measuring the estrogen receptor binding activity of a sample, obtained from the breast cancer tumor from said patient, thereby providing the estrogen receptor binding profiles in the presence and absence of one or more added phosphatases, estradiol and optionally a drug using the markers as listed in Table 1 selected from the group comprising SEQ ID NO 1 to 154; and,
(b) predicting from said estrogen receptor binding profiles the response of said patient to drug therapy.

8. A method for individualized estrogen receptor therapy of a patient diagnosed with an estrogen receptor related disease comprising the steps of:
(a) measuring the binding activity of the estrogen receptor, obtained from a sample from said patient, generating an binding activity profile, said binding activity profile comprising the binding activity of the estrogen receptor from said patient on one or more immobilized co-regulator proteins or functional parts thereof in the presence and absence of one or more added phosphatases,
(b) measuring the binding activity of the estrogen receptor, obtained from a sample from said patient, generating an binding activity profile, said binding activity profile comprising the binding activity of the estrogen receptor from said patient on one or more immobilized co-regulator proteins or functional parts thereof in the presence and absence of added estradiol,
(c) measuring on the basis of a sample the binding of the estrogen receptor, obtained from a breast cancer tumor from said patient, generating a binding profile, said binding profile comprising the binding of the estrogen receptor from said patient on one or more immobilized co-regulator proteins or functional parts thereof in the presence and absence of added estradiol and one or more added phosphatases, and,
(d) predicting from said binding activity profiles the response and optimum dose of said estrogen receptor therapy to said patient.

9. Method according to claim 8 wherein between step (a) and (c) an additional step of measuring the binding activity of the estrogen receptor, obtained from a sample from said patient, generating an activity profile, said activity profile comprising the activity of the estrogen receptor from said patient on one or more immobilized co-regulator proteins or functional parts thereof in the presence and absence of added estrogen receptor therapy drug is included.

10. A method for individualized endocrine therapy of a patient diagnosed with an endocrine related disorder comprising the steps of:
(a) measuring the binding activity of the nuclear receptor, obtained from a sample from said endocrine disease from said patient, generating a binding activity profile, said binding activity profile comprising the binding activity of the nuclear receptor from said patient on one or more immobilized co-regulator proteins or functional parts thereof in the presence and absence of one or more added phosphatases,
(b) measuring the binding activity of the nuclear receptor, obtained from a sample from said endocrine disease from said patient, generating a binding activity profile, said binding activity profile comprising the binding activity of the nuclear receptor from said patient on one or more immobilized co-regulator proteins or functional parts thereof in the presence and absence of added ligand of said nuclear receptor,
(c) measuring the binding of the nuclear receptor, obtained from a sample from said endocrine disease from said patient, generating a binding activity profile, said binding activity profile comprising the binding of the nuclear receptor from said patient on one or more immobilized co-regulator proteins or functional parts thereof in the presence and absence of added estradiol and one or more added phosphatases, and,
(d) predicting from said binding activity profiles the response and optimum dose of said endocrine therapy to said patient.

11. Method according to claim 10 wherein between step (a) and (c) an additional step of measuring the binding activity of the nuclear receptor, obtained from a sample from said endocrine related disorder from said patient, generating a binding activity profile, said binding activity profile comprising the binding activity of the nuclear hormone receptor from said patient on one or more immobilized co-regulator proteins or functional parts thereof in the presence and absence of added endocrine therapy drug is included.

12. The method according to claim 10 or 11 wherein the nuclear receptor consists of the androgen receptor, constitutive androstane receptor, estrogen receptor, farnesoid X receptor, glucocorticoid receptor, liver X receptor, peroxisome proliferator-activated receptor, progesteron receptor, retinoic acid receptor, thyroid receptor or vitamin D3 receptor.

13. The method according to any of claims 1 to 12, wherein said one or more added phosphatases are chosen from the protein phosphatase Mg²⁺- or Mn²⁺-dependent (PPM) family or an alkaline phosphatase.

14. Use of an array comprising a multitude of different immobilized proteins, peptides or peptide mimetics comprising estrogen receptor binding sites present in at least two peptide markers as listed in Table 1 selected from the group comprising SEQ ID NO 1 to 154 for carrying out the method of any of claims 1 to 13.

15. A method according to any of the previous claims allowing the determination of the basal activity levels of the nuclear receptor for use in calibration or normalization or in patient specific calibration or normalization (intra-assay).


SEQUENCE LISTING (154 sequences)

SEQ ID NO: 1 - protein, 11 aa, Artificial Sequence
  Peptide
MLSRLLKEHQA

SEQ ID NO: 2 - protein, 23 aa, Artificial Sequence
  Peptide
TVAASPAASGAPTLSRLLEAGPT

SEQ ID NO: 3 - protein, 24 aa, Artificial Sequence
  Peptide
GNLVPDAASKHKQLSELLRGGSGS

SEQ ID NO: 4 - protein, 23 aa, Artificial Sequence
  Peptide
GMVQDAASKHKQLSELLRSGSSP

SEQ ID NO: 5 - protein, 23 aa, Artificial Sequence
  Peptide
EDRAGRGPLPSPSLSELLASTAV

SEQ ID NO: 6 - protein, 23 aa, Artificial Sequence
  Peptide
PLGSAMLRPNPILARLLRAHGAP

SEQ ID NO: 7 - protein, 23 aa, Artificial Sequence
  Peptide
KYGEMPVDKAKAPLRELLRERAE

SEQ ID NO: 8 - protein, 23 aa, Artificial Sequence
  Peptide
PLVSQNNEQGSTLRDLLTTTAGK

SEQ ID NO: 9 - protein, 23 aa, Artificial Sequence
  Peptide
TTSPTAATTQNPVLSKLLMADQD

SEQ ID NO: 10 - protein, 24 aa, Artificial Sequence
  Peptide
HGEDFSKVSQNPILTSLLQITGNG

SEQ ID NO: 11 - protein, 23 aa, Artificial Sequence
  Peptide
LLAGPRSEAGHLLLQKLLRAKNV

SEQ ID NO: 12 - protein, 24 aa, Artificial Sequence
  Peptide
SDGDSKYSQTSHKLVQLLTTTAEQ

SEQ ID NO: 13 - protein, 24 aa, Artificial Sequence
  Peptide
PSSHSSLTERHKILHRLLQEGSPS

SEQ ID NO: 14 - protein, 23 aa, Artificial Sequence
  Peptide
ASKKKESKDHQLLRYLLDKDEKD

SEQ ID NO: 15 - protein, 21 aa, Artificial Sequence
  Peptide
TSGPQTPQAQQKSLLQQLLTE

SEQ ID NO: 16 - protein, 24 aa, Artificial Sequence
  Peptide
GQSRLHDSKGQTKLLQLLTTKSDQ

SEQ ID NO: 17 - protein, 24 aa, Artificial Sequence
  Peptide
STHGTSLKEKHKILHRLLQDSSSP

SEQ ID NO: 18 - protein, 23 aa, Artificial Sequence
  Peptide
EPVSPKKKENALLRYLLDKDDTK

SEQ ID NO: 19 - protein, 23 aa, Artificial Sequence
  Peptide
QRGPLESKGHKKLLQLLTCSSDD

SEQ ID NO: 20 - protein, 23 aa, Artificial Sequence
  Peptide
MHGSLLQEKHRILHKLLQNGNSP

SEQ ID NO: 21 - protein, 23 aa, Artificial Sequence
  Peptide
EQLSPKKKENNALLRYLLDRDDP

SEQ ID NO: 22 - protein, 23 aa, Artificial Sequence
  Peptide
MAGENHQWQGSILYNMLMSAKQT

SEQ ID NO: 23 - protein, 23 aa, Artificial Sequence
  Peptide
FSGKDHPRQGSILYSMLTSAKQT

SEQ ID NO: 24 - protein, 24 aa, Artificial Sequence
  Peptide
GEDHPRQGSILYSLLTSSKQTHVA

SEQ ID NO: 25 - protein, 23 aa, Artificial Sequence
  Peptide
SPSQGAASRPAILYALLSSSLKA

SEQ ID NO: 26 - protein, 23 aa, Artificial Sequence
  Peptide
TFEVAEAPVPSILKKILLEEPSS

SEQ ID NO: 27 - protein, 23 aa, Artificial Sequence
  Peptide
EVLEPWSPAAQGRLTRVLLTAST

SEQ ID NO: 28 - protein, 23 aa, Artificial Sequence
  Peptide
PEIQGIFDRDPDTLLYLLQQKSE

SEQ ID NO: 29 - protein, 23 aa, Artificial Sequence
  Peptide
VDSVPKGKQDSTLLASLLQSFSS

SEQ ID NO: 30 - protein, 23 aa, Artificial Sequence
  Peptide
PATSPKPSVASSQLALLLSSEAH

SEQ ID NO: 31 - protein, 23 aa, Artificial Sequence
  Peptide
RNNIKQAANNSLLLHLLKSQTIP

SEQ ID NO: 32 - protein, 23 aa, Artificial Sequence
  Peptide
KNSKLNSHQKVTLLQLLLGHKNE

SEQ ID NO: 33 - protein, 27 aa, Artificial Sequence
  Peptide
PVSPQDFSFSKNGLLSRLLRQNQDSYL

SEQ ID NO: 34 - protein, 23 aa, Artificial Sequence
  Peptide
RSWARESKSFNVLKQLLLSENCV

SEQ ID NO: 35 - protein, 23 aa, Artificial Sequence
  Peptide
EKDSPRLTKTNPILYYMLQKGGN

SEQ ID NO: 36 - protein, 23 aa, Artificial Sequence
  Peptide
SSQNHIPIEPDYKFSTLLMMLKD

SEQ ID NO: 37 - protein, 23 aa, Artificial Sequence
  Peptide
GTGGLSAVSSGPRLRLLLLESVS

SEQ ID NO: 38 - protein, 23 aa, Artificial Sequence
  Peptide
AGMLQGGASGEALLTHLLSDISP

SEQ ID NO: 39 - protein, 22 aa, Artificial Sequence
  Peptide
ENSNQNRELMAVHLKTLLKVQT

SEQ ID NO: 40 - protein, 23 aa, Artificial Sequence
  Peptide
DSELLVSPREGSSLHKLLTLSRT

SEQ ID NO: 41 - protein, 26 aa, Artificial Sequence
  Peptide
DGTPPPQEAEEPSLLKKLLLAPANTQ

SEQ ID NO: 42 - protein, 21 aa, Artificial Sequence
  Peptide
PAPEVDELSLLQKLLLATSYP

SEQ ID NO: 43 - protein, 21 aa, Artificial Sequence
  Peptide
AEFSILRELLAQDVLCDVSKP

SEQ ID NO: 44 - protein, 23 aa, Artificial Sequence
  Peptide
SVVQHADGEKSNVLRKLLKRANS

SEQ ID NO: 45 - protein, 23 aa, Artificial Sequence
  Peptide
ESRPQNANYPRSILTSLLLNSSQ

SEQ ID NO: 46 - protein, 23 aa, Artificial Sequence
  Peptide
LSEDIVDGHERAMLKRLLRIDSK

SEQ ID NO: 47 - protein, 23 aa, Artificial Sequence
  Peptide
TQSAVMDGAPDSALRQLLSQKPM

SEQ ID NO: 48 - protein, 23 aa, Artificial Sequence
  Peptide
HSLFEAKGDVMVALEMLLLRKPV

SEQ ID NO: 49 - protein, 23 aa, Artificial Sequence
  Peptide
GHGPTLKAYQEGRLQKLLKMNGP

SEQ ID NO: 50 - protein, 23 aa, Artificial Sequence
  Peptide
ESIYIHNIKDMKLLKTLLDIPAN

SEQ ID NO: 51 - protein, 23 aa, Artificial Sequence
  Peptide
GQRNISTLSTIQKLPRLLVASSS

SEQ ID NO: 52 - protein, 23 aa, Artificial Sequence
  Peptide
LQNLKNLGESATLRSLLLNPHLR

SEQ ID NO: 53 - protein, 23 aa, Artificial Sequence
  Peptide
VYPRPPSKTYRGAFQNLFQSVRE

SEQ ID NO: 54 - protein, 23 aa, Artificial Sequence
  Peptide
SVQPPRSISPSALQDLLRTLKSP

SEQ ID NO: 55 - protein, 23 aa, Artificial Sequence
  Peptide
TADPEKRKLIQQQLVLLLHAHKS

SEQ ID NO: 56 - protein, 24 aa, Artificial Sequence
  Peptide
TADPEKRKLIQQQLVLLLHAHKCQ

SEQ ID NO: 57 - protein, 24 aa, Artificial Sequence
  Peptide
TADPEKRKLIQQQLVLLLHAHKSQ

SEQ ID NO: 58 - protein, 22 aa, Artificial Sequence
  Peptide
SLRASQEQIEALLESSLRQAQQ

SEQ ID NO: 59 - protein, 18 aa, Artificial Sequence
  Peptide
MPVKRSLKLDGLLEENSF

SEQ ID NO: 60 - protein, 19 aa, Artificial Sequence
  Peptide
PHKASRHLDSYEFLKAILN

SEQ ID NO: 61 - protein, 23 aa, Artificial Sequence
  Peptide
LLTGTPLQNTVEELFSLLHFLEP

SEQ ID NO: 62 - protein, 23 aa, Artificial Sequence
  Peptide
QYQVALSASPLTSLPRLLDAKGI

SEQ ID NO: 63 - protein, 23 aa, Artificial Sequence
  Peptide
KNGNQLREYQLEGLNWLLFNWYN

SEQ ID NO: 64 - protein, 23 aa, Artificial Sequence
  Peptide
DLRGFAAQFIKQKLPDLLRSYID

SEQ ID NO: 65 - protein, 23 aa, Artificial Sequence
  Peptide
IPPTLAMNPQAQALRSLLEVVVL

SEQ ID NO: 66 - protein, 23 aa, Artificial Sequence
  Peptide
RAKSYHNLDAFVRLIALLVKHSG

SEQ ID NO: 67 - protein, 23 aa, Artificial Sequence
  Peptide
ELTKPMQILYKGTLRVLLVLLHD

SEQ ID NO: 68 - protein, 23 aa, Artificial Sequence
  Peptide
KPMQILYKGTLRVLLVLLHDFPE

SEQ ID NO: 69 - protein, 23 aa, Artificial Sequence
  Peptide
LSRILDVAQDLKALSMLLNGTPF

SEQ ID NO: 70 - protein, 23 aa, Artificial Sequence
  Peptide
DMVGVAQTGSGKTLSYLLPAIVH

SEQ ID NO: 71 - protein, 22 aa, Artificial Sequence
  Peptide
QFPLPKNLLAKVIQIATSSSTA

SEQ ID NO: 72 - protein, 22 aa, Artificial Sequence
  Peptide
EFPQPKNLLNSVIGRALGISHA

SEQ ID NO: 73 - protein, 23 aa, Artificial Sequence
  Peptide
SPLKPGTVSQQALQNLLRTLRSP

SEQ ID NO: 74 - protein, 23 aa, Artificial Sequence
  Peptide
QVSVLPEGGETPLFKQFFKNWRD

SEQ ID NO: 75 - protein, 23 aa, Artificial Sequence
  Peptide
RQLRRDKRDARRELKLLLLGTGE

SEQ ID NO: 76 - protein, 23 aa, Artificial Sequence
  Peptide
GLAKHLLSGLGDRLSRLLRRERE

SEQ ID NO: 77 - protein, 23 aa, Artificial Sequence
  Peptide
PLHLAVEAQAADVLELLLRAGAN

SEQ ID NO: 78 - protein, 23 aa, Artificial Sequence
  Peptide
LHLAVIHQHEPFLDFLLGFSAGT

SEQ ID NO: 79 - protein, 23 aa, Artificial Sequence
  Peptide
QWVSSLNEREQELHNLLEVVSQS

SEQ ID NO: 80 - protein, 23 aa, Artificial Sequence
  Peptide
WHNNVLHTHLVFFLPHLLNQPFS

SEQ ID NO: 81 - protein, 23 aa, Artificial Sequence
  Peptide
LAKFSPYLGQMINLRRLLLSHIH

SEQ ID NO: 82 - protein, 23 aa, Artificial Sequence
  Peptide
ALYVDSLFFLRGRLDQLLRHVMN

SEQ ID NO: 83 - protein, 23 aa, Artificial Sequence
  Peptide
LSGVMLTDVSPEPLQALLERASA

SEQ ID NO: 84 - protein, 23 aa, Artificial Sequence
  Peptide
DLVFDESGITDDQLLALLPSLSH

SEQ ID NO: 85 - protein, 23 aa, Artificial Sequence
  Peptide
TLHLERLAYLHARLRELLSELGR

SEQ ID NO: 86 - protein, 23 aa, Artificial Sequence
  Peptide
HLHLETFKAVLDGLDVLLAQEVR

SEQ ID NO: 87 - protein, 24 aa, Artificial Sequence
  Peptide
VSSMAGNTKNHPMLMNLLKDNPAQ

SEQ ID NO: 88 - protein, 23 aa, Artificial Sequence
  Peptide
HTDSLELLQLQQKLLWLLYDLGH

SEQ ID NO: 89 - protein, 23 aa, Artificial Sequence
  Peptide
HPVFQQESFTRQVLWKLLKVVKF

SEQ ID NO: 90 - protein, 23 aa, Artificial Sequence
  Peptide
PRLKKWKGVRWKRLRLLLTIQKG

SEQ ID NO: 91 - protein, 23 aa, Artificial Sequence
  Peptide
GAGRASESSYMSSLRILLDILEE

SEQ ID NO: 92 - protein, 23 aa, Artificial Sequence
  Peptide
SQSTFNNPRPGQLGRLLPNQNLP

SEQ ID NO: 93 - protein, 23 aa, Artificial Sequence
  Peptide
KKKGQGVIDKDSLGPLLLQALDG

SEQ ID NO: 94 - protein, 23 aa, Artificial Sequence
  Peptide
QRGPLESKGHKKLLQLLTSSSDD

SEQ ID NO: 95 - protein, 23 aa, Artificial Sequence
  Peptide
HGSQNRPLLRNSLDDLLGPPSNA

SEQ ID NO: 96 - protein, 23 aa, Artificial Sequence
  Peptide
RKPENGSRETSEKFKLLFQSYNV

SEQ ID NO: 97 - protein, 23 aa, Artificial Sequence
  Peptide
QLKEETLQQQAQQLYSLLGQFNS

SEQ ID NO: 98 - protein, 23 aa, Artificial Sequence
  Peptide
LVSPAMREAPTSLSQLLDNSGAP

SEQ ID NO: 99 - protein, 23 aa, Artificial Sequence
  Peptide
PVNKDVTLTSPLLVNLLQSDISA

SEQ ID NO: 100 - protein, 22 aa, Artificial Sequence
  Peptide
TTITAANFIDVIITRQIASDKD

SEQ ID NO: 101 - protein, 23 aa, Artificial Sequence
  Peptide
MGQVPRTHRLITLADHICQIITQ

SEQ ID NO: 102 - protein, 23 aa, Artificial Sequence
  Peptide
MGQVPRTHRLITLADHISQIITQ

SEQ ID NO: 103 - protein, 23 aa, Artificial Sequence
  Peptide
GHSFADPASNLGLEDIIRKALMG

SEQ ID NO: 104 - protein, 23 aa, Artificial Sequence
  Peptide
SSTGSTQFPYNPLTMRMLSSTPP

SEQ ID NO: 105 - protein, 23 aa, Artificial Sequence
  Peptide
SKNFYFNYKRRHNLDNLLQQHKQ

SEQ ID NO: 106 - protein, 23 aa, Artificial Sequence
  Peptide
APGVKGHQRVVTLAQHISEVITQ

SEQ ID NO: 107 - protein, 23 aa, Artificial Sequence
  Peptide
QAVQEHASTNMGLEAIIRKALMG

SEQ ID NO: 108 - protein, 23 aa, Artificial Sequence
  Peptide
SKNFYFNYKKRQNLDEILQQHKL

SEQ ID NO: 109 - protein, 23 aa, Artificial Sequence
  Peptide
QETLPRDSPDLLLLLRLLALGQG

SEQ ID NO: 110 - protein, 23 aa, Artificial Sequence
  Peptide
YVLHITKQRNKNALLRLLPGLVE

SEQ ID NO: 111 - protein, 23 aa, Artificial Sequence
  Peptide
IASEGKAEERYKKLEDLLEKSFS

SEQ ID NO: 112 - protein, 21 aa, Artificial Sequence
  Peptide
FRPIIGDVDIAGLLGDMLLLR

SEQ ID NO: 113 - protein, 23 aa, Artificial Sequence
  Peptide
DSVRKGKQDSTLLASLLQSFSSR

SEQ ID NO: 114 - protein, 23 aa, Artificial Sequence
  Peptide
KDLRCYGVASSHLKTLLKKSKVK

SEQ ID NO: 115 - protein, 23 aa, Artificial Sequence
  Peptide
KDLRSYGVASSHLKTLLKKSKVK

SEQ ID NO: 116 - protein, 23 aa, Artificial Sequence
  Peptide
GSEIENLLERRTVLQLLLGNPNK

SEQ ID NO: 117 - protein, 23 aa, Artificial Sequence
  Peptide
SEIENLLERRTVLQLLLGNPTKG

SEQ ID NO: 118 - protein, 23 aa, Artificial Sequence
  Peptide
GSDVHQDSIVLTYLEGLLMHQAA

SEQ ID NO: 119 - protein, 23 aa, Artificial Sequence
  Peptide
RSWARESKSFNVLKQLLLSENSV

SEQ ID NO: 120 - protein, 23 aa, Artificial Sequence
  Peptide
GGDSALSGELSASLPGLLSDKRD

SEQ ID NO: 121 - protein, 23 aa, Artificial Sequence
  Peptide
SPKTRESSLKRRLFRSMFLSTAA

SEQ ID NO: 122 - protein, 23 aa, Artificial Sequence
  Peptide
EEDADTKQVYFYLFKLLRKSILQ

SEQ ID NO: 123 - protein, 23 aa, Artificial Sequence
  Peptide
QDPPATMELAVAVLRDLLRYAAQ

SEQ ID NO: 124 - protein, 23 aa, Artificial Sequence
  Peptide
LFRDISMNHLPGLLTSLLGLRPE

SEQ ID NO: 125 - protein, 23 aa, Artificial Sequence
  Peptide
SQGLKHTESWEQELHSLLASLHT

SEQ ID NO: 126 - protein, 23 aa, Artificial Sequence
  Peptide
ESWEQELHSLLASLHTLLGALYE

SEQ ID NO: 127 - protein, 23 aa, Artificial Sequence
  Peptide
SPFFLQSLHGDGPLRLLLLPSIH

SEQ ID NO: 128 - protein, 23 aa, Artificial Sequence
  Peptide
VHPPNRSAPHLPGLMSLLRLHGS

SEQ ID NO: 129 - protein, 23 aa, Artificial Sequence
  Peptide
TSSRSRRELYSLLLALLLAPSPR

SEQ ID NO: 130 - protein, 23 aa, Artificial Sequence
  Peptide
ELRNMVSSFRVSELQVLLGFAGR

SEQ ID NO: 131 - protein, 22 aa, Artificial Sequence
  Peptide
SFNPSDKEIMTFQLKTLLKVQV

SEQ ID NO: 132 - protein, 23 aa, Artificial Sequence
  Peptide
QAFISEIGIEASDLSSLLEQFEK

SEQ ID NO: 133 - protein, 23 aa, Artificial Sequence
  Peptide
WPWDGELNADDAILRELLDESQK

SEQ ID NO: 134 - protein, 23 aa, Artificial Sequence
  Peptide
QQARLYENLPPAALRKLLRAEPE

SEQ ID NO: 135 - protein, 23 aa, Artificial Sequence
  Peptide
RLDSGMAFAGDEVLVQLLSGDKA

SEQ ID NO: 136 - protein, 23 aa, Artificial Sequence
  Peptide
ILYSGPSNKSVDVLAGLLLRRME

SEQ ID NO: 137 - protein, 23 aa, Artificial Sequence
  Peptide
EVRLERRASSGQALWLLLPARSS

SEQ ID NO: 138 - protein, 23 aa, Artificial Sequence
  Peptide
SPALQTPSLSSGQLPPLLIPTDP

SEQ ID NO: 139 - protein, 21 aa, Artificial Sequence
  Peptide
PPQEAEEPSLLKKLLLAPANT

SEQ ID NO: 140 - protein, 23 aa, Artificial Sequence
  Peptide
SPPEKDSGLLDSVLDTLLAPSGP

SEQ ID NO: 141 - protein, 23 aa, Artificial Sequence
  Peptide
SDTLPEVSAIPISLDGLLFPRPS

SEQ ID NO: 142 - protein, 23 aa, Artificial Sequence
  Peptide
EPSTVPGTPPPKKFRSLFFGSIL

SEQ ID NO: 143 - protein, 23 aa, Artificial Sequence
  Peptide
SIIQSPELMMDRHLDQLLMSAIY

SEQ ID NO: 144 - protein, 23 aa, Artificial Sequence
  Peptide
SEALLQLQFDDEDLGALLGNSTD

SEQ ID NO: 145 - protein, 23 aa, Artificial Sequence
  Peptide
FHEREGRPYSRRDFLQLFAPRSQ

SEQ ID NO: 146 - protein, 19 aa, Artificial Sequence
  Peptide
FQERAGKPYSQPSFLKLFG

SEQ ID NO: 147 - protein, 23 aa, Artificial Sequence
  Peptide
TALLYSKRLITYRLRHLLRARSD

SEQ ID NO: 148 - protein, 23 aa, Artificial Sequence
  Peptide
FVNLYTRERQDRLAVLLPGRHPS

SEQ ID NO: 149 - protein, 23 aa, Artificial Sequence
  Peptide
SLTESRREERVLQLLRLLNPSLE

SEQ ID NO: 150 - protein, 23 aa, Artificial Sequence
  Peptide
GSHDLLYQEFLPLLPNLLQGLNM

SEQ ID NO: 151 - protein, 23 aa, Artificial Sequence
  Peptide
LVAMMSLEDNKHALYQLLAHPNF

SEQ ID NO: 152 - protein, 23 aa, Artificial Sequence
  Peptide
DDEEPIPESSELTLQELLGEERR

SEQ ID NO: 153 - protein, 23 aa, Artificial Sequence
  Peptide
RDLGDSHPVLYQSLKDLLEYEGN

SEQ ID NO: 154 - protein, 23 aa, Artificial Sequence
  Peptide
LLGRSIQPEQVQRLTELLENDPS